(19) United States
(12) Patent Application Publication
(10) Pub. No.: US 2022/0220448 A1
(43) Pub. Date: Jul. 14, 2022

(54) INDUCED HUMAN COLITIC ORGANOIDS

(71) Applicant: The Cleveland Clinic Foundation, Cleveland, OH (US)

(72) Inventors: Emina Huang, Cleveland, OH (US); Samaneh Kamali, Lakewood, OH (US)

(21) Appl. No.: 17/609,957

(22) PCT Filed: May 13, 2020
(86) PCT No.: PCT/US2020/032624

§ 371 Date: Nov. 9, 2021

Related U.S. Application Data
(60) Provisional application No. 62/848,151, filed on May 15, 2019.

(51) Int. Cl. C12N 5/071; G01N 33/50; A61K 49/00

(52) U.S. Cl. CPC C12N 5/0679; G01N 33/5044; G01N 33/5088; A61K 49/0008; C12N 2506/23; C12N 2501/119; C12N 2501/16; C12N 2501/415; C12N 2501/15; C12N 2501/11; C12N 2500/38; C12N 2506/45

(57) ABSTRACT

Provided herein are compositions, systems, kits, and methods that employ a colitic induced human colitic organoid (iHCO) that has both an epithelial compartment and mesenchymal compartment, and provides at least one feature (e.g., leaky epithelial barrier) of IBD patient tissue (e.g., ulcerative colitis or Crohn's disease tissue). In certain embodiments, such iHCO's are employed in vitro or in vivo to screen candidate IBD treating compounds (e.g., to determine effectiveness for a particular patient who was the source of the original colonic fibroblasts used to generate the iHCO).

The present application claims priority to U.S. Provisional application Ser. No. 62/848,151 filed May 15, 2019, which is herein incorporated by reference.

### STATEMENT REGARDING FEDERAL FUNDING

This invention was made with government support under CA142808, CA157663, CA214300 and CA237304 awarded by the National Institutes of Health. The government has certain rights in the invention.

### FIELD

Provided herein are compositions, systems, kits, and methods that employ an induced human ulcerative-colitis derived organoid (iHUCO) that has both epithelial and mesenchymal compartment, and provides at least one feature (e.g., leaky epithelial barrier) of IBD patient tissue (e.g., ulcerative colitis or Crohn's disease tissue). In certain embodiments, such iHUCOs are employed in vitro or in vivo to screen candidate IBD treating compounds (e.g., to determine effectiveness for a particular patient who was the source of the original colonic fibroblast used to generate the iHUCO).

### BACKGROUND

Ulcerative colitis (UC), one of the two principal types of inflammatory bowel disease (IBD), is a chronic and debilitating inflammatory condition of the colonic mucosa that usually begins in young adulthood [1]. Although the precise etiology is unknown, UC likely results from complex pathologic interactions that involve genetic predisposition, immune activity, and the colonic microenvironment. The exposure of the epithelium to soluble inflammatory mediators secreted by cells in this microenvironment, including immune cells and stromal fibroblasts, is thought to play an essential early role in the development and progression of UC [2, 3].

The colonic epithelium is a highly dynamic tissue that in health, regenerates every 3 to 5 days. Regulation of gene expression in this complex process is controlled by several mechanisms, including the Wnt signaling pathway, which is responsible for maintaining epithelial homeostasis and an intact epithelial barrier [9]. Although canonical Wnt signaling (β-catenin dependent) is the most thoroughly investigated and potentially dominant Wnt pathway in intestinal development and homeostasis [10, 11], non-canonical Wnt signaling (β-catenin independent) has been noted to contribute to both development and disease pathogenesis [12, 13].

Current experimental models do not adequately recapitulate the complexity or etiology of clinical UC. No cell lines model the disease phenotype. Recent in vitro models, including epithelial organoids, focus solely on the epithelial compartment and do not address the role of the microenvironment such as the mesenchyme in disease progression [14, 15]. Common in vivo rodent models employing toxins such as dextran sodium sulfate (DSS) have advantages but still incompletely recapitulate the disease [16]. No patient-derived models are available. Until we have adequate models, dissection of UC disease pathogenesis, targeted intervention, and precision treatment will not be achieved.

### SUMMARY

Provided herein are compositions, systems, kits, and methods that employ an induced human colitic organoid (iHUCO) that has both an epithelial and mesenchymal compartment, and provides at least one feature (e.g., leaky epithelial barrier) of IBD patient tissue (e.g., ulcerative colitis or Crohn's disease tissue). In certain embodiments, such iHUCOs are employed in vitro or in vivo to screen candidate IBD treating compounds (e.g., to determine effectiveness for a particular patient who was the source of the original colonic fibroblasts used to generate the iHUCO).

In some embodiments, provided herein are compositions comprising: an induced human colitic organoid (iHUCO), wherein the iHUCO comprises an epithelial compartment and mesenchymal compartment, and provides at least one feature of IBD patient tissue. In certain embodiments, the at least one feature comprises a leaky epithelial barrier. In other embodiments, the at least one feature is selected from the group consisting of: disorganization of the epithelium compartment, elevated expression of CXCL8, and elevated expression of CXCR1. In additional embodiments, the compositions further comprises growth media, a hydrogel, and/or one or more candidate IBD treating compounds. In some embodiments, the composition is located in vitro. In further embodiments, the IBD tissue comprises ulcerative colitis tissue. In additional embodiments, the IBD tissue comprises Crohn's disease tissue.

In certain embodiments, provided herein are compositions comprising: an induced human colitic spheroid. In some embodiments, the compositions further comprise growth media, a hydrogel, and/or one or more candidate IBD treating compounds.

In particular embodiments, provided herein kits or systems comprising: a) an induced human colitic organoid (iHUCO) and/or an induced human colitic spheroid; and b) a candidate IBD treating compound (e.g., a known IBD treating compound or one that is not yet known to work, such as from a compound library).

In some embodiments, provided herein are methods of screening candidate IBD treating compounds in vitro comprising: a) contacting an induced human colitic organoid (iHUCO) with a candidate IBD treating compound, wherein the iHUCO comprises an epithelial compartment and mesenchymal compartment, and provides at least one feature of IBD patient tissue; and b) determining if the contacting causes the at least one feature of IBD patient tissue to be more like non-IBD tissue. In other embodiments, the iHUCO is derived from a colonic fibroblast from a human subject with IBD. In further embodiments, the contacting is found to cause the at least one feature of IBD patient tissue to be more like non-IBD tissue, and wherein the method further comprises treating the subject with the candidate IBD treating compound.

In certain embodiments, the IBD patient tissue comprises Ulcerative Colitis patient tissue or Crohn's disease patient tissue.

In some embodiments, provided herein are methods of screening candidate IBD treating compounds in vivo comprising: a) implanting a composition into a test animal (e.g., mouse or rat), wherein the composition comprises: an induced human colitic organoid (iHUCO) and/or an induced human colitic spheroid (iHS); and b) administering a candidate IBD treatment compound to the test animal. In further embodiments, the methods further comprise: c) examining the iHUCO and/or iHS for changes (e.g., to see if they are more like non-IBD type tissue). In further embodiments, the composition comprises a hydrogel surrounding the iHUCO and/or iHS.

In particular embodiments, provided herein are methods of generating induced human colitic organoid (iHUCO) in comprising: a) contacting a population of colonic fibroblasts from a human subject with inflammatory bowel disease (IBD) with: i) one or more expression vectors encoding iPSC reprogramming factors, or ii) RNAs encoding the iPSC reprogramming factors; to generate induced pluripotent stem cells (iPSCs), b) contacting the iPSCs with a transforming growth factor beta pathway agonist to generate definitive endoderm; c) contacting the definitive endoderm with a WNT signaling pathway agonist, a WNT/FGF signaling pathway agonist, a FGF signaling pathway agonist, or a combination thereof, thereby generating induced human colitic spheroids; and d) culturing the spheroids in culture media with at least one of the following: Respondin1, Noggin, EGF, retinoic acid, and a BMP inhibitor, thereby generating induced human colitic organoids (iHUCOs).

In certain embodiments, the IBD is ulcerative colitis or Crohn's disease. In other embodiments, the transforming growth factor beta pathway agonist comprises Activin A. In certain embodiments, the FGF signaling pathway agonist is FGF4. In other embodiments, the WNT pathway agonist is WNT3a.

### DESCRIPTION OF THE FIGURES

The patent or application file contains at least one drawing executed in color. Copies of this patent or patent application publication with color drawings will be provided by the Office upon request and payment of the necessary fee.

FIGS. 1A-L: In vitro patterning of induced human colonic organoids recapitulates the primary tissues. (A) Schematic representation of iHUCO generation protocol followed by the immunofluorescence staining of the key proteins in each stage of development: FB (B) expressing α-SMA (green) and lack of expression of CK19 (red); iPSC (C) expressing Tra-1-60 (green) and Oct-4 (red), DE (D) expressing SOX17 (green) and FOXA2 (red), SPH (E) expressing CDX2 (green) confirming their intestinal identity; and iHUCO (F) expressing CK19 (red) in epithelium and Vimentin (green) in the mesenchyme. Nuclei in all IF images counterstained with DAPI (blue). (G1-G4) Representative H&E of iHNO and UC iHUCO with simple monolayer epithelium in iHNO (G1) vs. stratified epithelium in iHUCO (G2) and the matched primary tissues (G3, G4) that exhibit similar morphological patterns, respectively. (H) Epithelial thickness in non-IBD and UC organoids and primary tissues. (I1-I4) Representative Ki67 immunohistochemistry for iHNO (I1) iHUCO (12) and primary non-IBD (13) UC (I4) tissues. (J) Percentages of cells positive for Ki67 in all groups. (K1-K4) Representative Alcian blue-Periodic acid-schiff stain (AB-PAS) of iHNO (K1) and iHUCO (K2) and the matched primary tissues (K3, K4). (L) Number of goblet cells in non-IBD/UC organoids and primary tissues. IF scale bar, 25 um. IHC scale bar, 40 um. N=3 of non-IBD (blue) and UC (red). ***(p<0.001), ****(p<0.0001).

FIGS. 2A-H: The iHUCOs demonstrate aberrant adherens junction formation in the epithelium. (A, C) Representative immunohistochemistry of β-catenin (A) and E-cadherin (C) demonstrating the difference in cellular localization among iHNO and iHUCO as well as the matched primary tissues. (B, D) Percentages of the cells demonstrating expression of β-catenin (B) and E-cadherin (D) in organoid and primary tissue; separated by cellular compartment: plasma membrane-only (Mem), and cytoplasm+nucleus (Cyt+Nuc). (E) Representative IHC for RhoA demonstrating increased cytoplasmic and membrane expression of RhoA in iHUCO vs. iHNO, and the UC primary tissues vs. non-IBD (F) Percentage of the cells positive for RhoA in plasma membrane-only (Mem), and cytoplasmic (Cyt) compartments. (G) Percentage of cells expressing Wnt pathway target proteins regulating sternness (G1) and proliferation (G2), respectively. Results demonstrate the difference in expression patterns of these proteins in iHUC vs. iHN organoids. (H) TOPflash assay on non-IBD and UC spheroids demonstrating fold decrements of Wnt3A activity in UC vs. non-IBD. PC=positive control of the assay. Scale bar, 40 um. N=3 of non-IBD (blue) and UC (red). **(p<0.01), ***(p<0.001), ****(p<0.0001).

FIGS. 3A-G: Transcriptome-wide analysis of iHUCOs recapitulates the colitic signatures (A) Principle component analyses (PCA) was conducted for non-IBD and UC iPSCs, DE, SPHs, and organoids (N=3 each group). The first principal component (PC1) accounts for 92.5% of the variations in the data. (B, left) Spearman ranking was applied to cluster samples based on their similarity in order to generate a heatmap with the highest level of correlation (dark blue). (B, right) Venn diagram of differentially expressed genes in iHCOs vs. SPHs, SPHs vs. DE, and DE vs. iPSCs in UC and non-IBD (C) Differentially expressed genes in iHUCOs vs. SPHs (shown in yellow in Venn diagram) were applied to conduct a functional network analysis (Cytoscape) (Shannon, P., et al., Genome Res, 2003. 13(11): p. 2498-504.), highlighting the key features of iHUCOs. (D) Curated heatmaps based on the gene ontology (GO) terms highlighted in panel C including inflammation/immune response (top), and wound healing (bottom); UC iHCOs demonstrate an increase in the expression of these genes compared to SPHs. (E) Differentially expressed genes in iHUCOs vs. SPHs were subjected to GO analysis; the enriched GO terms are represented as REVIGO (Supek, F., et al., PLoS One, 2011. 6(7): p. e21800) scatterplots. The terms extend along X-axis based on similarity in the type of biological process (semantic space X); color differences indicate different types of enriched functions (p<0.001). Each circle represents a unique GO term. Circle size corresponds to the number of the genes associated with unique GO term. (F) Graphs of RPKM values for N=3 of UC vs. non-IBD SPHs. These graphs list a number of important genes regulating canonical and non-canonical Wnt pathways. The difference in fold-RPKM confirms higher levels of non-canonical Wnt activation in UC and an increase of canonical Wnt activation in non-IBD SPHs. (G) RPKM of the gene sets belonging to the highlighted GO terms in panel E were applied to list the top 50 expressed genes as a curated heatmap.

FIGS. 4A-F: The iHUCOs recapitulate the transcriptome of colitic stroma and epithelium (A) Dendrogram of the gene sets hierarchically clustered based on Canberra distance. Parental fibroblasts and all 24 samples in different stages of development were included in the analysis. Parental fibroblasts shared the highest level of the similarity with organoids compared to the other stages of development. (B) Ingenuity Pathway Analysis (IPA) was applied to conduct a comparison analysis in iHUCOs (orange) vs. UC fibroblasts (red). Positive z-scores indicate activated pathways, and the negative z-scores indicate down-regulation of the pathways in each group (C) Differentially expressed genes in iHUCOs vs. fibroblasts were analyzed applying gene ontology (GO) analysis, and the enriched terms are presented as REVIGO scatterplots. GO terms are grouped in arbitrary 2-dimensional space based on semantic similarity; the difference in colors is based on the range of the p-values. (D) Log₂ (fold change) of a subset of genes exclusive to UC epithelium extracted from GO terms related to cell junction and epithelium development in panel C. (E) GSEA analysis summary in iHUCOs (orange) vs. UC fibroblasts (red) and UC (red) vs. non-IBD (green) fibroblasts. NES and FDR-q values represent the significance of the highlighted functions. (F) Log₂ (fold change) of the highly significant genes belonging to the top 5 GO terms highlighted in UC vs. non-IBD fibroblasts. *(p<0.01), **(p<0.01), ***(p<0.001), ****(p<0.0001).

FIGS. 5A-M: CXCL8 receptor signaling: an inflammatory mediator in iHUCOs. (A) Representative vimentin-positive (VIM; green) and CK19-negative (red) immunofluorescence staining of non-IBD and UC organoid-derived mesenchyme. (B) Summarized percentages of cells positive for VIM, α-SMA (fibroblast markers), and CK19 (epithelium marker). (C) Representative cytokine arrays on the parental fibroblasts and organoid-derived mesenchyme with the quantification of three chemokines of interest, GRO-α (green), GRO-α+β+γ (blue), and CXCL8 (orange) in UC vs. non-IBD. (D-F) Representative immunofluorescence dual staining for CXCR1 (red) and CXCL8 (green) expressed in epithelium and mesenchyme of iHNO and iHUCO. (G, H) Summarized percentages of cells positive for CXCR1, CXCL8 and both (overlap), in the epithelium and mesenchyme of iHNO and iHUCO. (I) Representative immunofluorescence staining for E-cadherin (green) and β-catenin (red) co-localization in iHN (I1) and iHUC (12) organoids. (J, K) Summarized percentages of cells positive for both E-cadherin and β-catenin proteins in plasma membrane (Mem), and cytoplasm+nucleus (Cyt+Nuc) of non-IBD and UC organoids. All nuclei are stained with DAPI (blue). (L) Immunohistochemistry for Claudin-1 in iHNO (L1) and iHUCO (L2). (M) Epithelial barrier permeability measurements in real-time for non-IBD (blue) and UC (orange) organoids over 15 hours. Immunofluorescence scale bar, 25 um. IHC Scale bar, 40 um. N=3 each; except for barrier studies, N=1. ****(p<0.0001).

FIGS. 6A-U: Repertaxin attenuates the progression of the colitic phenotype in iHUCOs in vitro. (A, B) Representative immunofluorescence co-localization expression of CXCR1 (red) and CXCL8 (green) expressed in epithelium and mesenchyme of iHNO and iHUCO in the absence (A) or presence of repertaxin (B). (C, D) Summarized percentages of cells positive for CXCR1, CXCL8 and both, in the epithelium and mesenchyme of non-IBD and UC iHCOs, after treatment with repertaxin (20 μm) compared to vehicle (Ctrl). (E) Representative H&E of non-IBD and UC organoids in Ctrl compared to repertaxin treated (F). (G, H) Organoid mean diameter and epithelial thickness after 21 days of treatment with repertaxin compared to control. (I, M) Representative immunohistochemistry for β-catenin and E-cadherin in Ctrl organoids compared to repertaxin-treated (J, N); revealing altered cellular localization of the proteins after treatment (K, L, O, P) Summarized percentages of cells in non-IBD and UC organoids positive for β-catenin (K, L), and E-cadherin (O, P) according to cellular compartment: plasma membrane only (Mem) and cytoplasm+nucleus (Cyt+Nuc), with or without treatment with repertaxin. (Q, R) Representative immunohistochemistry for RhoA demonstrating the changes in cellular localization after treatment with repertaxin (R) compared to control (Q). (S, T) Quantification of the percentage of the cells in control vs. repertaxin-treated organoids, positive for RhoA in plasma membrane-only (Mem) and cytoplasm (Cyt). (U) Relative permeability of the epithelial barrier to 4 kDa dextran measured in real-time for untreated iHNOs (blue, N=11) vs. repertaxin-treated iHNOs (red, N=10) (U1) and untreated iHUCOs (orange, N=17) vs. repertaxin-treated iHUCOs (green, N=16) (U1) during 15 hours demonstrating a significant decrease in the epithelial barrier leakage in UC. IHC Scale bar, 40 um. IF Scale bar, 25 um. N=3 each; except for barrier studies, N=1. ns: not significant, **(p<0.01), ***(p<0.001), ****(p<0.0001).

FIGS. 7A-U: Repertaxin attenuates the progression of the colitic phenotype in iHUCOs in vivo. (A) Schematic representation of repertaxin study in vivo; spheroids encapsulated in TS-HA hydrogel beads were implanted subcutaneously in the dorsal flanks of immunocompromised NSG mice receiving daily injections of repertaxin vs. PBS (21 days). (B, C) Representative immunofluorescence dual-staining of CXCR1 (red) and CXCL8 (green) expressed in epithelium and mesenchyme of iHNO and iHUCO in Ctrl (B) or repertaxin-treated (C). (D, E) Summarized percentages of cells positive for CXCR1, CXCL8 and both, in the epithelium and mesenchyme of non-IBD and UC organoids, after treatment with repertaxin (20 mg/kg) compared to PBS (Ctrl). (F-G) Representative H&E of non-IBD and UC organoids harvested after 21 days for control (F) and repertaxin treatment (G). (H, I) Mean organoid diameter and epithelial thickness after 21 days of repertaxin treatment vs. Ctrl. (J, N) Representative immunohistochemistry for β-catenin and E-cadherin in non-IBD and UC organoids in Ctrl compared to (K, O) repertaxin-treated; revealing the effect of repertaxin injection on the cellular localization of both proteins (L, M, P, Q) Summarized percentages of cells in organoids positive for β-catenin (L, M), and E-cadherin (P, Q) with or without repertaxin treatment. Results reported for the plasma membrane only (Mem), and cytoplasm+nucleus (Cyt+Nuc) cellular compartments. (R, S) Representative immunohistochemistry of RhoA, highlighting the effect of repertaxin on the cellular localization of the protein in plasma membrane only (Mem), and cytoplasm (Cyt) compartments. (T, U) Summarized percentages of cells expressing RhoA in Ctrl vs. repertaxin-treated organoids for plasma membrane-only (Mem) and cytoplasm (Cyt). IHC Scale bar, 40 um. IF Scale bar, 25 um. N=3 each. ns=not significant, *(p<0.05), **(p<0.01), ***(p<0.001), ****(p<0.0001). FIG. 8, panels A-F: iHUCOs transplanted in omentum are responsive to the exogenous stiffness (A) Representative IHC for Ki67 staining in iHUCOs, demonstrating increased expression in intermediate and high elastic moduli (B) Percentage of the cells positive for Ki67 in all different moduli of non-IBD and UC transplanted organoids. (C) Representative IHC for pYAP1 staining in iHUCOs demonstrating increased expression in low elastic modulus (D) Percentage of the cells positive for pYAP1 in all different moduli of non-IBD and UC transplanted organoids. (E) Representative IHC for tYAP1 staining in iHUCOs demonstrating increased expression in intermediate and high elastic moduli (F) Percentage of the cells positive for tYAP1 in all different moduli of non-IBD and UC transplanted organoids.

FIGS. 9A-F: Resolution transcriptomic analysis of iHUCOs recapitulates colitic signatures. (A) UMAP consisting of 11 unique clusters among 44,185 nuclei of iHUCOs (N=3). (B) Marker plot highlighting the top expressed genes in each cluster of panel A; the size and color of the dots correlate with the abundance and the expression level, respectively. (C) Proportion plots of epithelial, stromal, and immune compartments in iHUCOs annotated by the subtypes in each compartment. (D) Representative immunohistochemistry (IHC) for HLA-A, and Limch1 proteins comparing the expression level in iHNOs and iHUCOs along with summarized percentages of cells positive for these proteins in the epithelium of organoids. (E) GO terms with the highest enrichment scores in iHUCOs; highlighting the importance of extracellular matrix organization in their colitic signature. (F) Representative immunohistochemistry (IHC) for Collagen I, and Periostin revealing a dramatic increase in their expression in iHUCOs vs. iHNOs (induced human non-IBD organoid). All nuclei are stained with DAPI (blue). Scale bar, 40 um. iHNO (N=5) and iHUCO (N=6) ****(p<0.0001). This work is based on single nuclear RNA-seq.

FIGS. 10A-D: High resolution transcriptomic analysis of iHNOs. (A) UMAP consisting of 16 unique clusters among 30,819 nuclei of iHNOs (N=3). (B) Marker plot highlighting the top expressed genes in each cluster of FIG. 10A; the size and color of the dots correlate with the abundance and the expression level, respectively. (C) Proportion plots of epithelial, and stromal compartments in iHUCOs annotated by the subtypes in each compartment. (D) Volcano plots highlighting the top expressed genes in Cycling TA, Stem, Myo FBs, and WNT5B+subtypes of iHUCOs vs. iHNOs. This work is based on single nuclear RNA-seq.

### DETAILED DESCRIPTION

Provided herein are compositions, systems, kits, and methods that employ the induced human colitic organoid (iHUCO) that has both an epithelial compartment and mesenchymal compartment, and provides at least one feature (e.g., leaky epithelial barrier) of IBD patient tissue (e.g., ulcerative colitis or Crohn's disease tissue). In certain embodiments, such iHUCO's are employed in vitro or in vivo to screen candidate IBD treating compounds (e.g., to determine effectiveness for a particular patient who was the source of the original colonic fibroblasts used to generate the iHUCO).

Provided herein, in certain embodiments, are methods for reprogramming of colonic fibroblasts isolated from UC patients to become iPSCs. Work conducted during development of embodiments herein demonstrated that the isolation of fibroblasts from UC and non-IBD colon is sufficient to retain the colonic identity in iHUCOs. Such iHUCOs include both epithelial and mesenchymal compartments, reflect the complexity and retains the colitic phenotype of the tissue of origin in vitro and in vivo. Such iHUCOs, therefore, not only facilitate strategies for personalized medicine (e.g., the patient with IBD can provide the original colonic fibroblast to growth iHUCOs as described herein) but also enables investigation of the mechanisms underlying the pathophysiology of human IBD and new therapeutic strategies in a less complex, more easily manipulated in vitro environment. One advantage of iHUCOs is that they preserve individual patient variation allowing patient-specific drug screening to be performed to identify the best compound or compounds to treat the patient.

Work conducted during development of iHUCO model embodiments herein revealed for that overexpression of CXCL8-CXCR1 in UC positively regulates the activation of RhoA protein, resulting in an increase of expression of activated RhoA and its mobilization to the plasma membrane as compared to the non-IBD organoid model, induced human non-IBD organoid (iHNO) and human tissues. Such work also demonstrated the functionality of the model via responses to chemical perturbation by the CXCR1/2 small molecule non-competitive inhibitor, repertaxin. Exposure of iHUCO cultures to repertaxin both in vitro and in vivo, demonstrated decreased expression of CXCL8 and CXCR1 and attenuated several aspects of the colitic phenotype, including a disorganized epithelium, aberrant proliferation, and persistence of a leaky epithelial barrier. Importantly, CXCL8 lacks a murine orthologue, which highlights the gap in the murine-based models and the further functional importance of the models herein in identifying the role of CXCL8-CXCR1-mediated signaling in colitis development and progression. Work conducted herein found that overexpression of the inflammatory CXCL8-CXCR1 axis in iHUCOs disrupts canonical Wnt signaling regulation, resulting in a dysregulated adherens junction pattern in iHUCOs epithelial cells. Furthermore, repertaxin, a CXCL8-CXCR inhibitor, significantly attenuated the progression of the colitic phenotype in iHUCOs.

Generating the iHUCO, described herein can start with a colonic fibroblast from a patient with IBD. Methods of generating iPSCs from the colonic fibroblast are described in Example 1 below and can be done using the reprogramming factors and methods known in the art. Differentiation such iPSCs to definitive endoderm, then spheroids, then final organoids can be performed as described in Example 1 below, as wells as in McCraken et al. (Nat Protoc, 2011. 6(12): p. 1920-8) and US Pat. Pub. 2017/0240866, both of which are herein incorporated by reference in their entireties.

### EXAMPLES

### Example 1

### Induced Patient-Derived Colitic Organoids Recapitulate Inflammatory Reactivity

Ulcerative colitis (UC) is a major type of inflammatory bowel disease (IBD), which affects millions of patients. The exact etiology of UC remains unknown, and no model exists that adequately recapitulates the complexity of the disease in vitro or in vivo. We developed an induced human ulcerative colitis-derived organoid (iHUCO) model using induced pluripotent stem cells (iPSCs) originating from fibroblasts harvested from the colons of UC patients and compared these to the induced human non-IBD organoid model (iHNO) derived from isolated non-IBD colonic fibroblasts. Both models contain the epithelial and mesenchymal compartments. Notably, the iHUCOs recapitulate histological and functional features of the primary colitic tissues, including the absence of neutral mucus secretion and a leaky epithelial barrier both in vitro and as in vivo xenografts, suggesting that intrinsic factors are sufficient to drive a UC phenotype after reprogramming. However, the iHNOs reveal features of normal colon, including mucus secretion and an intact epithelial barrier. Further, we used iHNO and iHUCO models to demonstrate that overexpression of the inflammatory mediator CXCL8 and its receptor CXCR1 led to dysregulated epithelial adherens junctions in iHUCO. As proof-of-principle, we show that CXCL8 receptor inhibition by repertaxin attenuates the progression of UC phenotypes both in vitro and in vivo. Our patient-derived model to recapitulate UC in vitro will generate new insights into the underlying pathogenesis of this complex disease.

### Results

### In Vitro Patterning of Induced Human Colonic Organoids Recapitulates the Primary Tissues

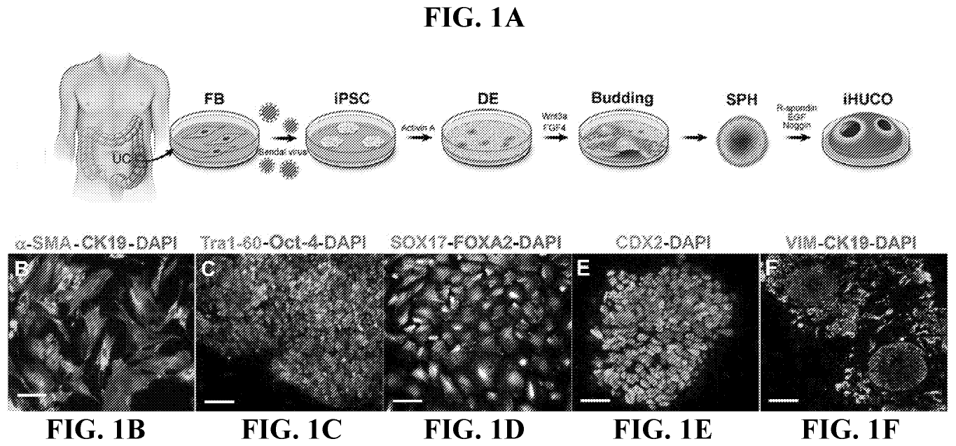

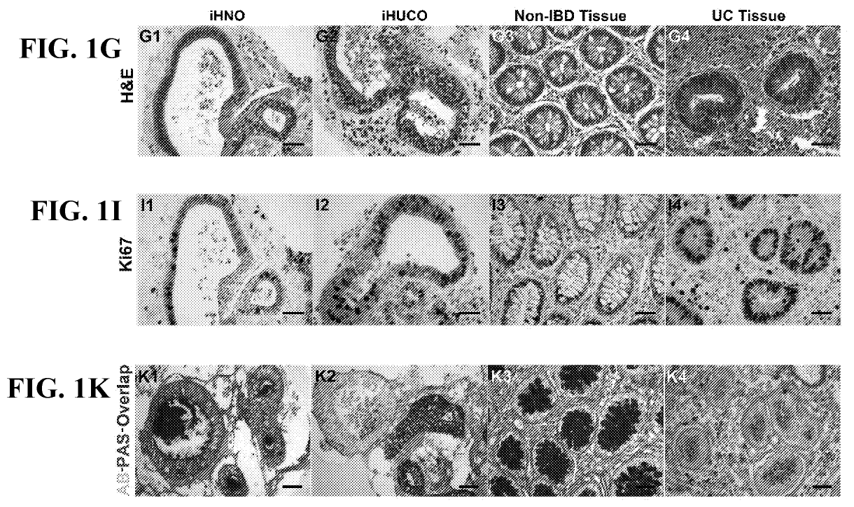

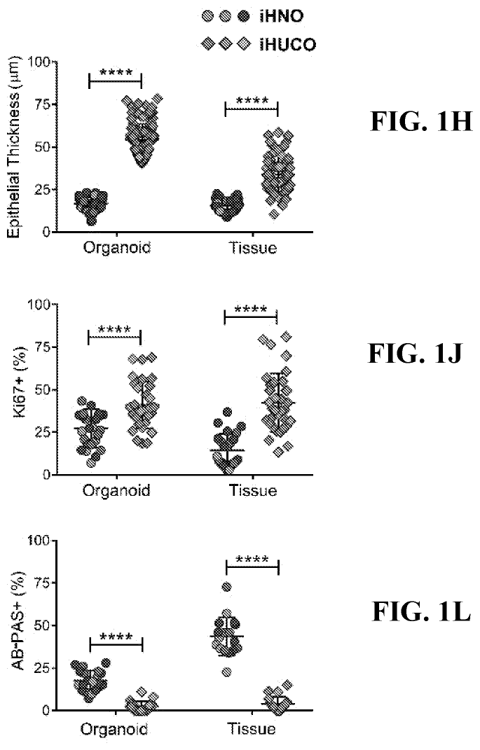

An exemplary schematic protocol for in vitro iHUCO patterning is illustrated in FIG. 1A. Fresh surgical specimens bearing inflamed tissues from the colon of patients with UC or the healthy colon were obtained, and fibroblasts were isolated and propagated as described previously [19]. The cell type was confirmed by visual inspection for spindle-shaped cells, positive immunofluorescence (IF) staining for smooth muscle actin, and the absence of cytokeratin 19 staining (FIG. 1B). Mycoplasma assay and short tandem repeat analysis were conducted to verify the absence of mycoplasma and the unique origin of each fibroblast isolate, respectively. Next, we reprogrammed the isolated fibroblasts to induced pluripotent stem cells (iPSCs) as described (STAR methods, which included transfecting the cells with four sendai viruses encoding Oct3/4, Sox2, c-Myc, and KLF4), generating both colitic and non-IBD iPSCs. Pluripotency of the generated iPSCs was confirmed at multiple levels, including an embryoid body-like appearance, immunofluorescent expression for proteins that indicate human pluripotency including Tra-1-60 and Oct-4 (FIG. 1C) and evidence of trilaminar potentiality. We applied McCracken et al.'s intestinal development protocol [18] for direct differentiation of iPSCs into definitive endoderm (using Activin A), validated by SOX17 and FOXA2 protein expression (FIG. 1D), followed by generation of intestinal spheroids (SPHs, CDX2 expression, FIG. 1E) using FGF4 and WNT3a. Spheroids were then cultured in Matrigel for 21 days (with Rspondin1, FGFR, and EGF) to generate organoids, including both epithelial and mesenchymal compartments (FIG. 1F).

Both iHNOs and iHUCOs were characterized by comparison to the matched primary tissues. Hematoxylin-eosin (H&E) staining of these organoids revealed distinct epithelial and mesenchymal domains with an interior lumen (FIG. 1G1, 1G2). IHNOs had a well-organized columnar epithelium representative of the healthy colonic mucosa (FIG. 1G1). In contrast, iHUCOs frequently had disorganized and multi-layered epithelium (FIG. 1G2). This observation was consistent with the pathology seen in large intestinal mucosa from patients with active UC in which crypts are morphologically more disorganized compared to non-IBD tissues [21] (FIG. 1G3, 1G4). Quantification of the epithelial thickness for N=3 of non-IBD (blue) and UC (red) organoids and their primary tissues supported our observations that UC epithelium in organoids and primary tissues are 2 to 3 times thicker of that in non-IBD (FIG. 1H). IHUCOs were also characterized for UC pathognomonic attributes.

Immunohistochemical (IHC) staining for the nuclear non-histone proliferation marker, Ki67, in the organoids and their primary tissues revealed more uniform cellular proliferation throughout the columnar epithelium of the iHNOs, similar to the primary non-IBD tissues (FIG. 1I1, 1I3). In contrast, regions of disorganized epithelium in iHUCOs and primary tissues had extensive and non-uniform epithelial proliferation with greater distribution (FIG. 1I2, 1I4), which was confirmed by quantification of epithelial Ki67. Ki67 was overexpressed up to 80% in iHUCOs and primary tissues; whereas it reached only 40% in the non-IBD condition (FIG. 1J). Our finding is consistent with the reported accelerated rate of epithelial cell turnover in colonic mucosa undergoing regeneration in patients with active UC [22].

The intestinal mucus layer secreted by goblet cells in the healthy mucosa includes both acidic and neutral mucin to protect the epithelial barrier from luminal bacterial penetration [23]. Therefore, we performed Alcian blue and Periodic acid-Schiff (AB-PAS) staining (FIG. 1K) to compare the mucus composition and the presence of goblet cells in the organoids and their primary tissues. For iHNOs, both acidic and neutral mucus secretions were present in the lumen along with a limited numbers of goblet cells (FIG. 1K1). As expected, goblet cells were present in all crypts of the differentiated non-IBD tissues (FIG. 1K3). In contrast, iHUCOs had either no mucus or only neutral mucus, suggesting they lacked acidic mucin secretory function (FIG. 1K2). Our observation was supported by a striking decrease in the number of goblet cells in the matched UC primary tissues (FIG. 1K4). Quantification of the number of goblet cells in all groups highlighted the loss of this cell type in iHUCOs and primary tissues (FIG. 1L). These data are consistent with the depletion of goblet cells and the mucus layer observed in the colonic mucosa of patients with UC [24].

Thus, we conclude that both non-IBD and UC adult human colonic fibroblasts can be reprogrammed to iPSCs, differentiated to intestinal spheroids and organoids. The iHUCOs phenocopy features of UC tissues, including disorganized/multi-layered epithelium, increased proliferation rate, and lack of mucus secretion.

### IHUCOs Demonstrate Aberrant Adherens Junction Formation in the Epithelium

The expression of CDX2 plays a crucial role in intestinal development, including cell fate determination, balancing proliferation with differentiation, and epithelial barrier formation [9, 25, 26]. As expected, uniform and strong expression of CDX2 restricted to the epithelium was observed in IHC staining of non-IBD colon tissues (FIG. S2A3). Following the same pattern, CDX2 was strongly expressed in the mature (STAR Methods) iHNOs (FIG. S2A1). In contrast, CDX2 expression was strikingly low in primary UC tissues and the corresponding organoids (FIG. S2A2, S2A4). Quantification for N=3 of non-IBD (blue) and UC (red) organoids and the primary tissues confirmed that CDX2 expression was significantly lower in both iHUCOs and UC tissues compared to non-IBD (FIG. S2B). This observation is consistent with previous reports of inflammation-related decrease in CDX2 for patients with active UC [27, 28].

Recently, SATB2 has been identified as a definitive marker of distal small intestine (ileum) and colonic epithelium in humans [10]. Similar to CDX2, IHC staining for SATB2 revealed less expression in UC than in non-IBD organoids and the primary tissues (Figure S2C). Although SATB2 expression was robust in non-IBD adult tissues, it was sharply lower in the epithelium of UC tissues (FIG. S2C3, S2C4). IHNOs with more of a fetal-like phenotype rather than adult colon [18, 29] expressed less SATB2 than the primary tissues. However, the reduced expression was greater in the UC organoids than non-IBD organoids (FIG. S2C1, S2C2). Quantification of SATB2 expression in both organoids and primary tissues confirmed the strong downregulation in UC (FIG. S2D).

In health, epithelial cells form a physical barrier within the gut lumen that protects the intestine from bacterial and inflammatory cell infiltration [30]. A dynamic combination of different apical junctions, including tight junctions and adherens junctions, between the epithelial cells maintains this homeostasis [30, 31]. In contrast, under pathological conditions such as UC, the balance in cellular junctions is disrupted, and the integrity of the epithelial barrier is compromised [6, 7]. This disruption results in an increase in para-cellular space, bacterial invasion, dysregulation of the immune response, and ultimately a leaky damaged epithelial barrier [2, 32, 33]. One of the main regulators of the intercellular junction and intestinal development is the multifunctional protein, β-catenin. Although the accumulation of β-catenin in the cytoplasm and its eventual translocation into the nucleus is essential for canonical Wnt pathway activation and subsequent expression of tight junction proteins, limited expression of β-catenin on the cell membrane co-localized with E-cadherin is a hallmark of adherens junction regulation [34]. An imbalance in the structural and cellular localization of β-catenin results in pathological conditions including dysregulation in intestinal development [6, 7, 34].

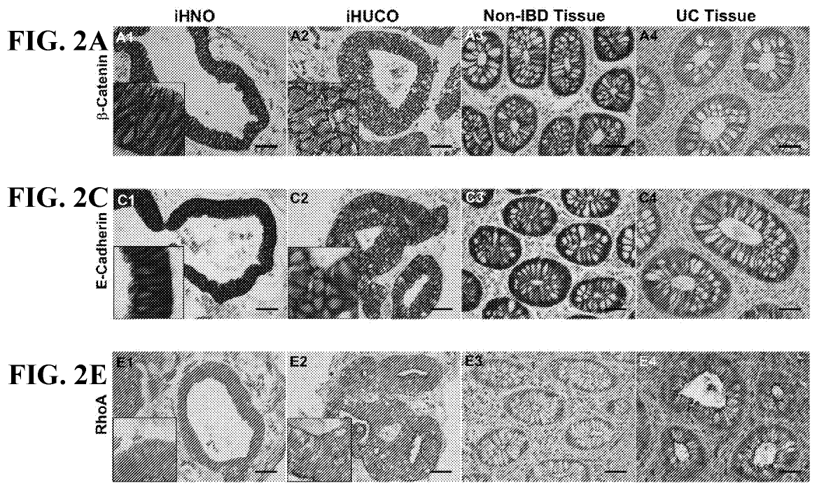

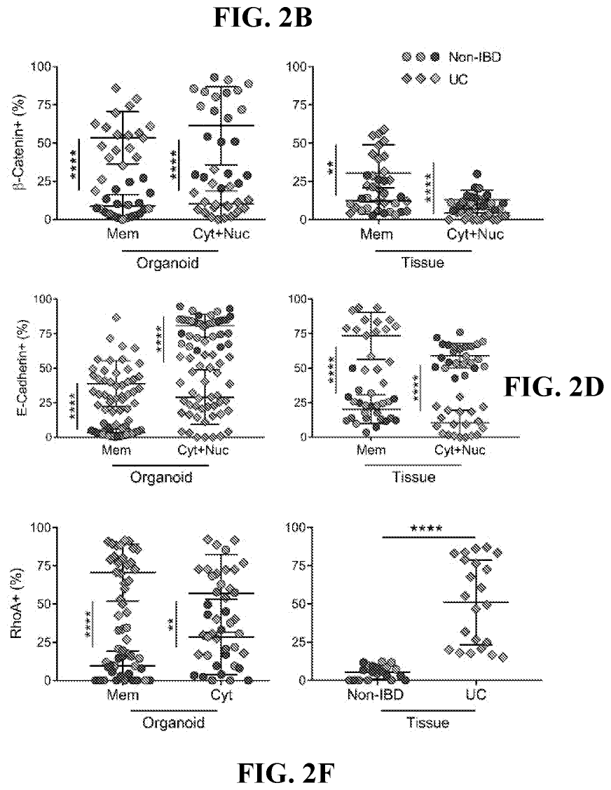

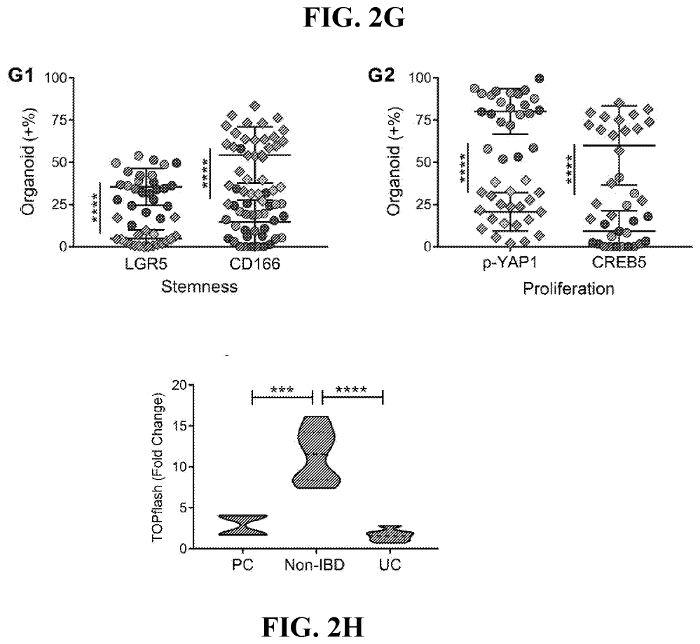

We performed IHC on organoids and their matched primary tissues to study the cellular localization of β-catenin (FIG. 2A). The iHNOs had strong membrane, cytoplasmic, and nuclear expression of β-catenin; suggesting a high degree of protein stability (FIG. 2A1). On the other hand, the iHUCOs lacked such strong expression, and the majority of the protein was limited to the plasma membrane (FIG. 2A2). Percentages of cells expressing β-catenin at the membrane-only and the combined cytoplasm and nucleus revealed that while the exclusive expression of β-catenin on the membrane of iHUCOs was approximately 3-fold that of iHNOs, the combined cytoplasmic and nuclear expression was significantly higher in iHNOs (FIG. 2B). This limited β-catenin expression was confirmed in UC and non-IBD primary tissues (FIGS. 2A3, 2A4, and 2B).

E-cadherin, the main component of the adherens junction complex, had a similar expression pattern as β-catenin in both organoids and primary tissues (FIG. 2C, 2D). Although E-cadherin was strongly expressed in the cytoplasm, nucleus, and plasma membrane of the iHNOs, cytoplasmic and nuclear expression were sharply and significantly lower in iHUCOs (FIGS. 2C1, 2C2, and 2D). Similarly, E-cadherin expression in all 3 sub-cellular components was greater in non-IBD and UC primary tissues (FIGS. 2C3, 2C4, and 2D).

RhoA is one of the dominant regulators of the adherens junction complex, playing roles in cell adhesion and cytoskeleton organization [35]. When activated, cytoplasmic (inactive) RhoA is translocated to the plasma membrane to regulate the formation of actin stress fibers (F-actin), downstream of the adherens junction dynamic [35]. IHC revealed significantly greater (up to 90%) RhoA expression in both the cytoplasm and plasma membrane of iHUCOs and the primary UC tissues than in iHNOs and their primary tissues (FIG. 2E2, 2E4, 2F).

We also examined the expression of additional Wnt target proteins involved in stemness and proliferation in the organoids (FIG. 2G). In situ hybridization for LGR5, a stem cell marker activated by Wnt/β-catenin pathway, revealed an approximately 8-fold greater percentage of cells expressing LGR5 in iHNOs than in iHUCOs (FIG. 2G1, S2E). In contrast, IHC staining for CD166, a stem cell marker regulated by the non-canonical Wnt pathway [36], demonstrated the opposite pattern with an average 4-fold greater percentage of cells in iHUCOs than in non-IBD organoids (FIG. 2G1, S2F). Under normal development, phosphorylation of yes-associated protein (p-YAP serine-127) is regulated by Hippo signaling to control organ growth and size [37]. As expected, IHC staining for p-YAP in iHNOs revealed high stability and retention of the protein in the cytoplasm. In contrast, iHUCOs showed an average of 4-fold less cytoplasmic p-YAP expression, confirming that the developmental pattern was dysregulated (FIG. 2G2, S2G). In addition, the expression of cyclic AMP-responsive element-binding protein 5 (CREB5), a proliferation marker regulated by non-canonical Wnt signaling pathway [38] was significantly higher in iHUCOs than iHNOs (FIG. 2G2, S2H). To further evaluate the Wnt/β-catenin activity, we performed a TOPflash functional assay (STAR methods) to compare the relative activity of Wnt/β-catenin signaling in vitro, in the non-IBD and UC spheroids as the principal developmental stage for canonical Wnt pathway activation [9, 39]. While the UC spheroids and positive control (PC) had ˜5-fold Wnt/β-catenin activity compared to the negative control of the assay (STAR methods) non-IBD spheroids showed up to a 15-fold more activity (FIG. 2H).

To summarize our in vitro findings, the expression of CDX2 and SATB2 in the organoids reflected their expression in the primary tissues; the expression of both markers was significantly lower in iHUCOs than iHNOs. Moreover, we found a similar pattern of expression between β-catenin and E-cadherin in the organoids, which was similar to patterns in their primary tissues. Although both proteins were strongly expressed in the membrane, cytoplasm, and nucleus subcellular components of the iHNOs, they were mainly limited to the plasma membrane in the iHUCOs. Furthermore, lower activity of Wnt/β-catenin signaling was present in iHUCO development that may resulted in an aberrant adherens junction pattern in epithelial cells.

To confirm the iHUCOs phenotype in vivo, we combined the recently reported omental transplantation protocol for PSC-derived organoids [40] with the biocompatible TS-HA hydrogel to encapsulate the non-IBD and UC organoids (STAR Methods) and transplanted one seeded bead in the omentum of host NSG mice (Figure S3A). After 90 days, the beads were collected and analyzed. H&E staining confirmed the colon formation in omentum (Figure S3B). The colitic phenotype, including stratified, shorter, and disorganized crypts (Figure S3B2), aberrant proliferation (Figure S3D2), and lack of acidic mucus accompanied by a limited number of goblet cells (Figure S3F2), was retained in iHUCOs-derived colon (Figure S3C, S3E, and S3G, respectively). As expected, the colon formed by iHNOs recapitulated normal colon, including monolayer epithelium (Figure S3B1, S3C), proliferation limited to the crypt base (S3D1, S3E), and the secretion of both acidic and neutral mucus as well as goblet cell generation (S3F1, S3G). In both non-IBD and UC organoids, the formed colon was also characterized for the expression of CDX2 and SATB2 proteins (Figure S3H-K). Similar to the in vitro pattern, expression of both proteins was low in UC but not in non-IBD organoid-derived colon. Next, we confirmed that the combined nuclear and cytoplasmic expression of β-catenin and E-cadherin was significantly lower in the colon formed by iHUCOs and that cytoplasmic expression of RhoA was higher in UC-derived colon. These data are consistent with the patterns seen in UC and non-IBD primary tissues (Figure S3L-Q).

The combination of all these observations both in vitro and in vivo confirmed dysregulation in the developmental process of iHUCOs. We observed features consistent with aberrant adherens junction formation in the UC epithelium. Our observation confirmed previous reports highlighting the classical role of E-cadherin as a canonical Wnt antagonist due to its role in tethering β-catenin on the plasma membrane as a part of the adherens junction complex [41].

Transcriptome-Wide Analysis of iHCOs Recapitulates Colitic Signatures

Transcriptome-Wide Analysis of iHUCOs Recapitulates the Colitic Signatures

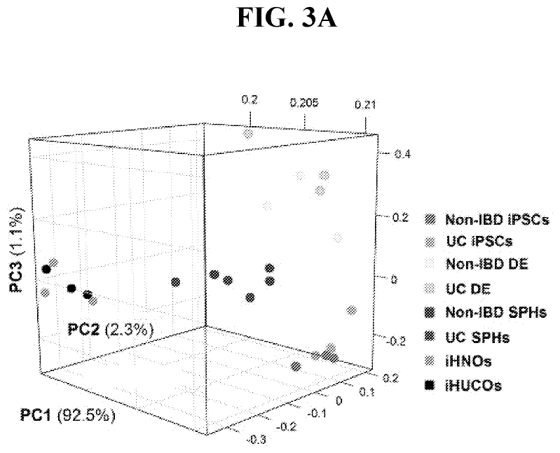

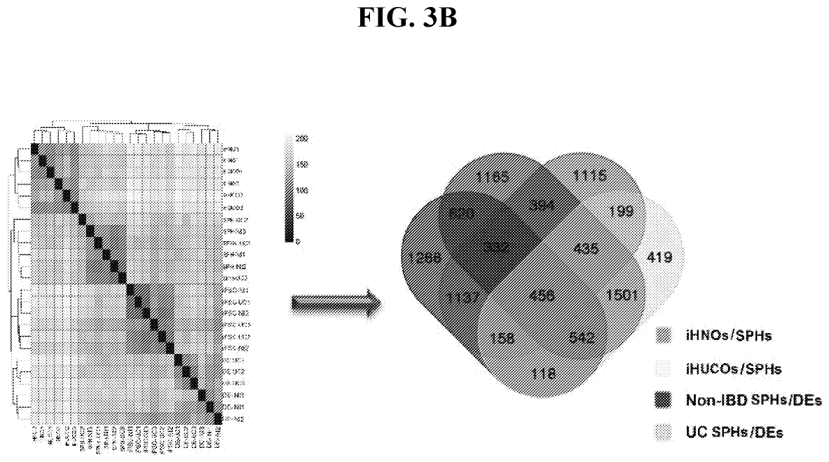

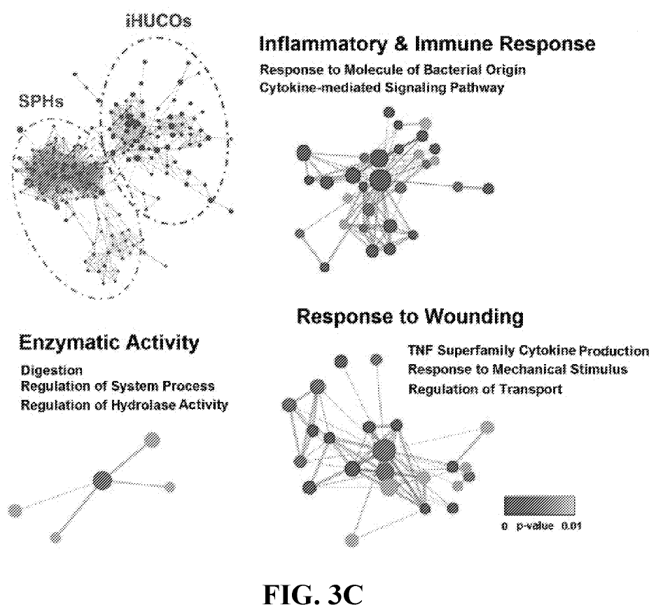

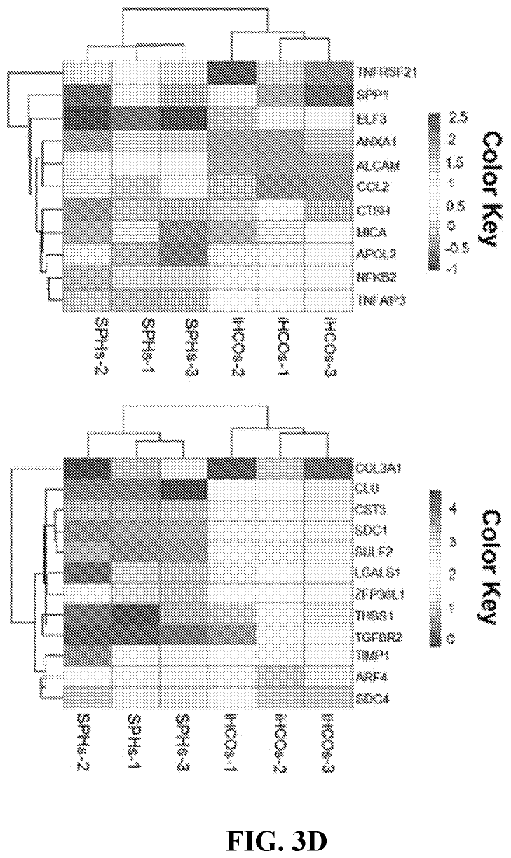

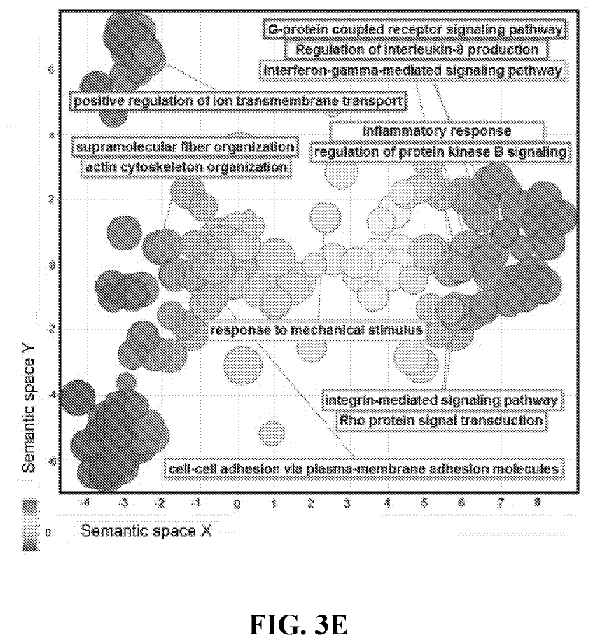

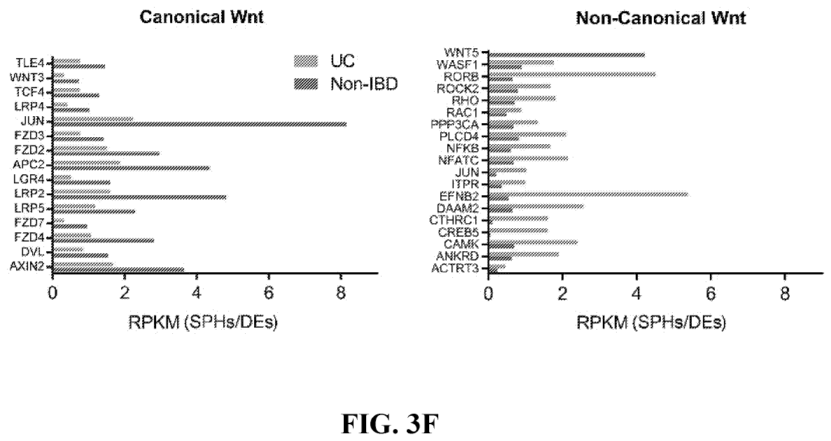

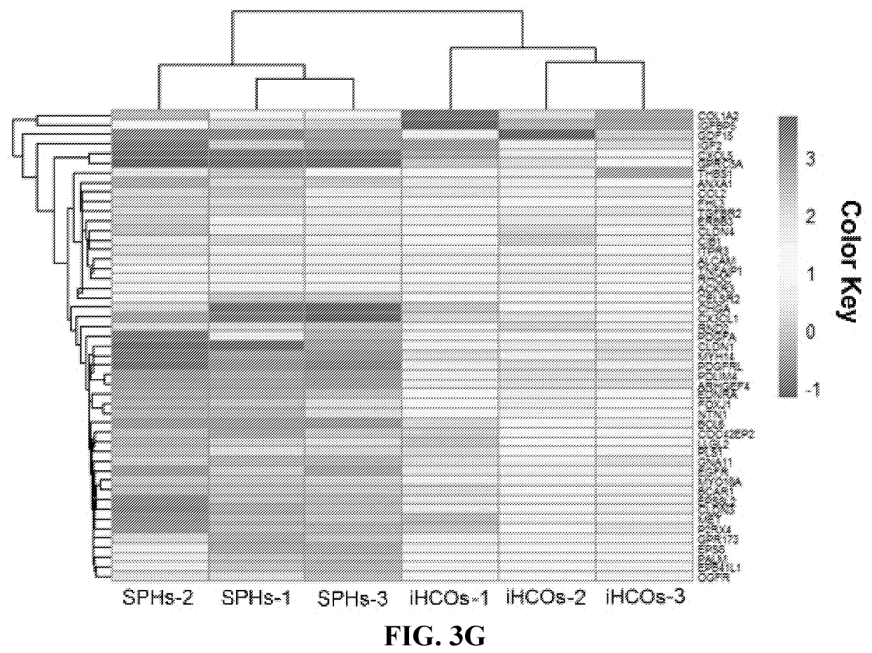

To investigate transcriptional features of our organoids, we conducted bulk RNA-Seq on both non-IBD and UC iPSCs, DE, spheroids, and organoids (N=3 for each group). Using the RNA-seq data, we compared the transcriptional activity during disease development with non-IBD development. Principal component analysis (PCA) revealed major variations in transcriptional abundance among all genes in the RNA-Seq dataset, and that the variation in the dataset was driven by the developmental stage (FIG. 3A). To improve our understanding of the similarities and differences between UC and non-IBD groups during intestinal development, we conducted a PCA among DE, spheroids, and organoids in both UC and non-IBD (N=3 for each) (Figure S4A). DE as the first stage of the intestinal development formed a distinct cluster causing a shift in the gene expression pattern between UC and non-IBD. Distinct from this pattern, subsequent progression in development to spheroids and organoids localized the non-IBD and UC groups closer to each other.

Unsupervised hierarchical clustering of the global gene expression data based on the Spearman rank correlation was performed (FIG. 3B, left). Consistent with PCA results, the groups segregated based on developmental stage rather than the disease status, and organoids formed a distinct cluster from DE and iPSCs but segregated closely with spheroids. Considering that the developmental stage was the main driver in gene expression, we sequentially calculated and compared the numbers of the differentially expressed genes in each stage of directed differentiation for UC and non-IBD. Consistent with our earlier observation of the global clustering, a Venn diagram of these differentially expressed genes (FIG. 3B, right) showed the greatest number of differentially expressed unique genes (1115 genes) in the progression from DE to spheroid in UC. We also identified 1501 genes in common between UC and non-IBD, and 419 unique genes in UC during progression from spheroid to organoid.

To explore the molecular states specific to iHUCO and immature spheroid, we conducted Gene Set Enrichment Analysis (GSEA) and determined the enriched terms by applying complex network analysis using Cytoscape [42] (FIG. 3C). Consistent with the nature of UC, significant functional terms (p<0.01) including inflammation and immune response, wound healing, defense and response to bacteria were identified in iHUCOs. We extracted the RPKM values for the key genes belonging to inflammation and immune response terms (FIG. 3D, top) and the response to wound healing term (FIG. 3D, bottom) to generate curated heatmaps of UC spheroids and organoids. For these specific gene subsets, iHUCOs clustered together as compared with spheroids and showed a significant increase in their transcriptome, suggesting a more mature colitic signature compared with spheroids.

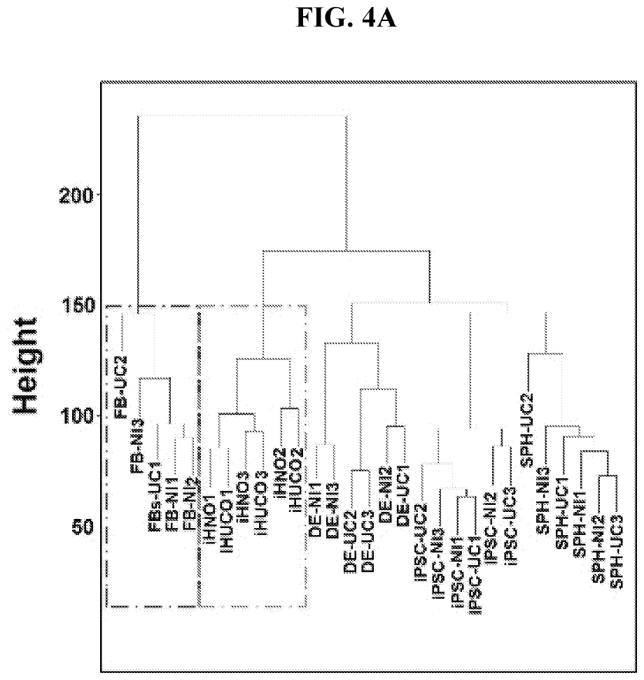

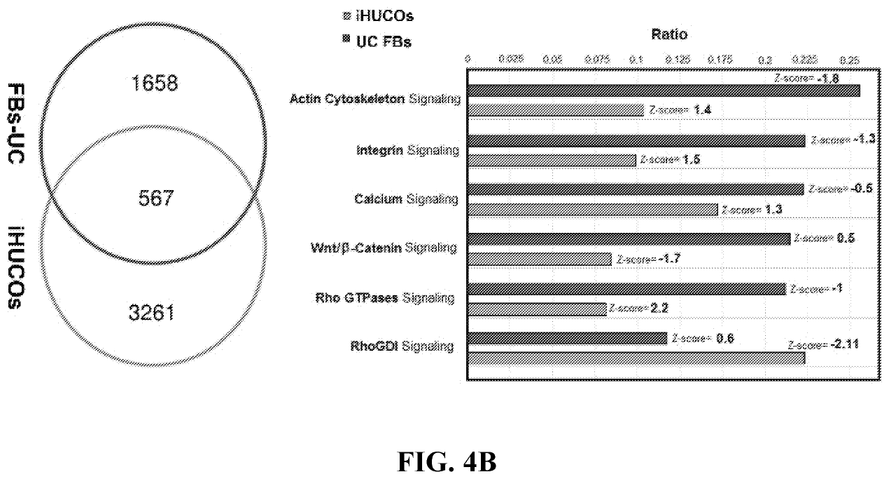

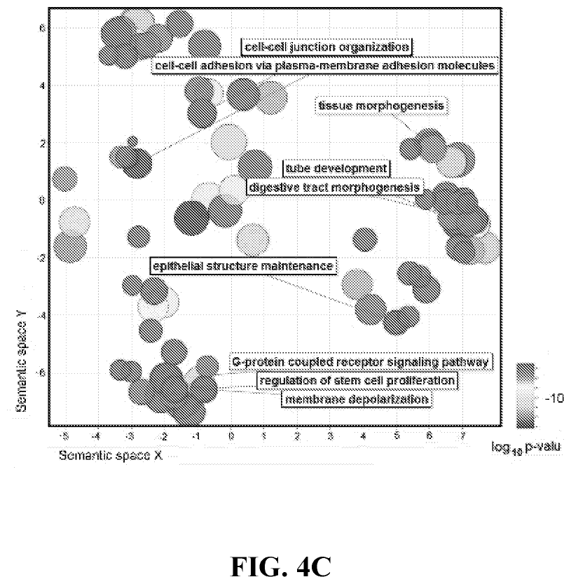

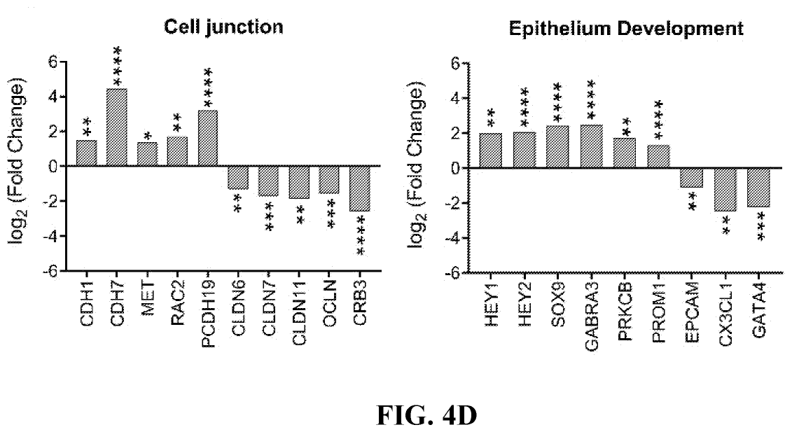

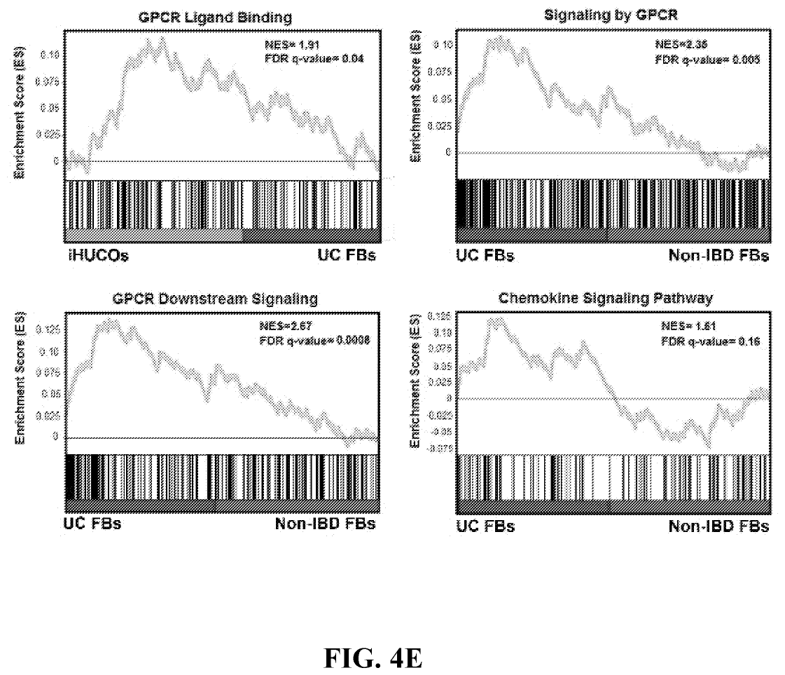

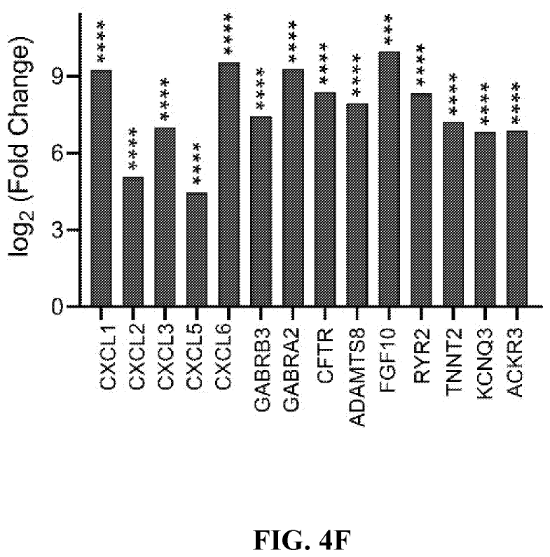

To identify dominant biological processes that were enriched in the iHUCOs, we applied Gene Ontology enrichment analysis tool (GOrilla) and Reduce and Visualize Gene Ontology (REVIGO) [43] (FIG. 3E). Enriched GO terms from a ranked list of the differentially expressed genes in the organoid and spheroid were reduced using REVIGO by clustering related terms semantically along the X-axis based on similarity in function. Highly significant (p<0.001) enriched terms including “actin cytoskeleton organization” and “fiber polymerization” clustered on the left, progressed to “response to mechanical stimuli” and “cell-cell adhesion,” and terminated on the right with signaling pathways including GPCR, regulation of interleukin-8 (CXCL8) production, and Rho protein signal transduction (FIG. 3E). Details for the highlighted GO terms including p-values, FDR q-values, and enrichment scores are shown in FIG. 4B.

The top 50 genes in iHUCOs, belonging to the highlighted GO terms in FIG. 3E, were applied to generate a curated heatmap (FIG. 3G). For these functions, the range of gene expression was mostly consistent in iHCOs (log₂ (RPKM)>1) and clustered together as compared with the expression in spheroids (FIG. 3G). Some of these genes, such as COL1A2 involved in the formation of very strong type I collagen fibers or GDF15 a secreted ligand of the TGF-beta superfamily involved in inflammation/acute injury, showed a highly significant increase in their transcriptome in organoids vs. spheroids (FIG. 3G). Consistent with our findings of the canonical Wnt signaling dysregulation in iHUCOs (FIG. 2), the enriched GO terms in the transcriptome of these organoids (FIG. 3E) mostly suggested the non-canonical Wnt signaling-induced downstream events, such as cytoskeleton organization, Rho protein signal transduction, and cell-cell adhesion via plasma membranes.

To further explore this observation, we extracted RPKM values of the key genes regulating the canonical and non-canonical Wnt signaling pathways for UC and non-IBD spheroids, as the principal developmental stage for Wnt pathway activation [9]. The UC spheroids had an upregulation pattern for the non-canonical Wnt signaling and a downregualtion pattern for the canonical Wnt signaling (FIG. 3F).

To also identify the dominant biological processes enriched in iHNOs, we applied GOrilla and REVIGO to the ranked list of the differentially expressed genes in iHNO and spheroids (Figure S4C1). Unlike UC, the highly significant GO terms included “cell fate specification” and “epithelial cell differentiation”. The role of GPCR signaling and its downstream effector cAMP-mediated signaling (involved in regulation of cell communication) were also significant (Figure S4C1, S4C2). Furthermore, we compared two unranked lists of the differentially expressed genes, iHUCO (target set) and iHNOs (background set), in GOrilla to visualize the enriched GO terms by REVIGO (FIG. 4SD1, 4SD2). Several GO terms involved in cell cycle progression and DNA repair were highlighted in this comparison; the highlighting of the aberrant cell cycle/proliferation in UC was consistent with our observations shown in FIGS. 11 and S3D.

To summarize, transcriptomic analyses of iHUCOs demonstrated their relevance and functional identity as an in vitro model for ulcerative colitis. Furthermore, the enriched molecular and biological processes in these organoids identified the roles of GPCR signaling, interleukin-8 (CXCL8), and downstream functions of non-canonical Wnt signaling such as Rho protein signal transduction in UC.

### IHUCOs Recapitulate the Transcriptome of Colitic Stroma and Epithelium

To confirm the colonic identity of iHNOs and iHUCOs at the transcriptome level, we used the list of genes reported by Múnera et al. [10] that were up-regulated in human colonic organoids (HCOs) and human intestinal organoids (HIOs) as well as adult colon and small intestine [10]. Heatmaps for these genes in all three stages of intestinal development were generated (Figure S5A). Although both iHNOs and iHUCOs had a log₂ (RPKM)≥1 for the majority of genes, the expression pattern of these genes were differed between non-IBD and UC according to the developmental stages (Figure S5A1, S5A2). We extracted the top 50 expressed genes in UC and non-IBD organoids and generated a Venn diagram to identify the highly expressed genes exclusive to non-IBD or UC (Figure S5B). Functional classification of these unique genes in PANTHER (Key Resources Table) highlighted the GO term “catalytic activity” and “binding/transport” as the main category in non-IBD and UC, respectively. We also generated curated heatmaps of the top 50 genes for both non-IBD and UC organoids (Figure S5C1, S5C2).

To examine the similarity between parental fibroblasts and each developmental stage, we conducted RNA-Seq on UC and non-IBD parental fibroblasts (GSE106119). Unsupervised hierarchical clustering based on the Canberra distance showed that parental fibroblasts shared the highest level of the similarity with the organoids compared with the other stages of development (FIG. 4A). Hypothesizing that this similarity originated from the mesenchymal compartment of organoids, we conducted two separate Ingenuity Pathway Analyses (IPA) for the genes differentially expressed in UC fibroblasts and organoids and applied the results of both analyses to conduct a comparison analysis in IPA (FIG. 4B). In this comparison, we first focused on the differentially expressed genes exclusive to iHUCOs (3261 genes, Venn diagram FIG. 4B) and identified the signaling pathways with opposing z-scores (opposing activation patterns) between the iHUCOs and their parental fibroblasts, shown as a bar graph in FIG. 4B. Similar to the results of REVIGO analysis in FIG. 3E, the signaling pathways related to the cell junction, cytoskeleton organization, and Rho GTPase were exclusively unregulated in iHUCOs whereas canonical Wnt signaling and Rho GDI signaling were downregulated.

We hypothesized that these pathways with the opposing z-scores between iHUCOs and fibroblasts originate from the epithelial compartment of the organoids. To test this hypothesis, we compared two unranked lists of the differentially expressed genes, iHUCOs (target set) and UC fibroblasts (background set), in GOrilla and visualized the highly significant GO terms (p-value<0.001) by REVIGO (FIG. 4C). The analysis confirmed that enriched GO terms including “tube development” and “epithelial structure maintenance” were mainly related to the epithelium development. Genes extracted from these GO terms were subjected to an additional analysis to identify those genes exclusive to colitic epithelium. The log₂ (fold change) values for a subset of these genes are shown in FIG. 4D according to two categories: i) the genes involved in cell-cell junction organization due to the importance of this GO term based on our previous analyses (FIG. 3), and ii) the genes with an exclusive role in UC epithelial development. Genes in the cell junction category, including CDH and CLDN, confirmed the regulation of adherens junction in UC epithelium, and genes differentially expressed during development of UC epithelium highlighted the role of notch and non-canonical Wnt signaling (FIG. 4D).

To also determine the signaling pathways in common between iHUCOs and fibroblasts, we considered the results of the IPA comparison analysis for the highly significant signaling pathways with the allied z-scores (similar activation pattern) between iHUCOs and UC FBs (Figure S5D). The signaling pathways such as “protein kinase A signaling” and “Tec kinase signaling” known in development, growth, and activation of immune cells were identified.

To further analyze the UC fibroblasts signature, we conducted GSEA on UC and non-IBD fibroblasts using the KEGG and Reactome datasets to identify the highly significant and enriched functional terms. The role of the GPCR signaling, chemokine signaling, and regulation of the GPCR downstream pathways were highlighted (FIG. 4E). The role of GPCR ligand binding as a highly significant term was also highlighted in conducted GSEA for iHUCOs and UC fibroblasts (FIG. 4E), confirming the significant role of GPCR signaling in iHUCOs, which was also enriched in our GOrilla/REVIGO analysis (FIGS. 3E, 4C). The highly significant genes belonging to the top 5 GO terms highlighted in UC and non-IBD fibroblasts are shown in FIGS. 4F, and S5E.

In summary, parental fibroblasts shared the highest level of similarity with the organoids. The differentially expressed genes in organoids and fibroblasts highlight the activation of signaling pathways such as the GPCR and Rho GTPases and the downregulation of canonical Wnt signaling in the UC epithelium. Furthermore, we confirmed the colitic signature of the UC parental fibroblasts at the transcription level and established the importance of GPCR downstream signaling and chemokine signaling in these fibroblasts as well as the mesenchymal compartment of iHUCOs.

CXCL8 Receptor Signaling: An Inflammatory Mediator in iHUCOs

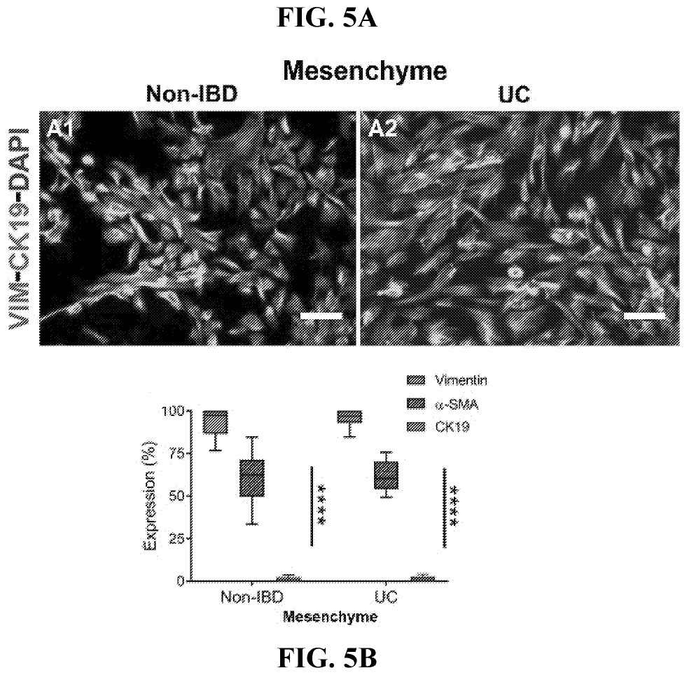

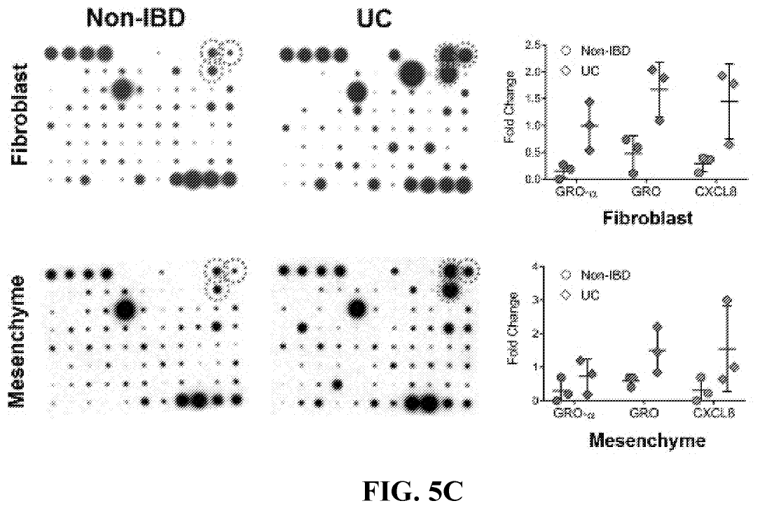

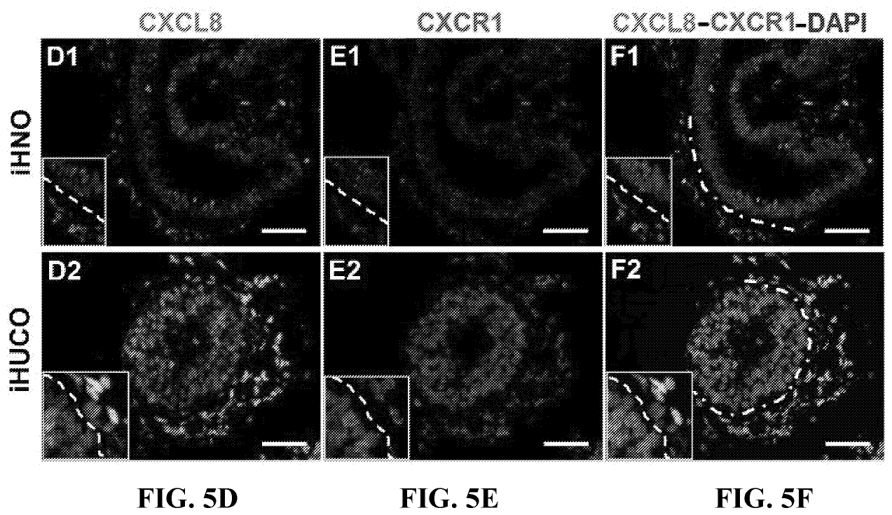

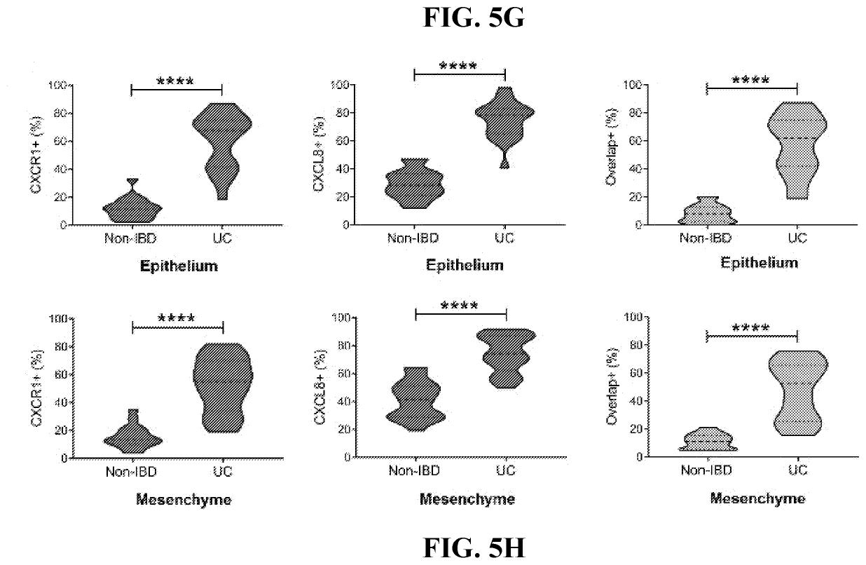

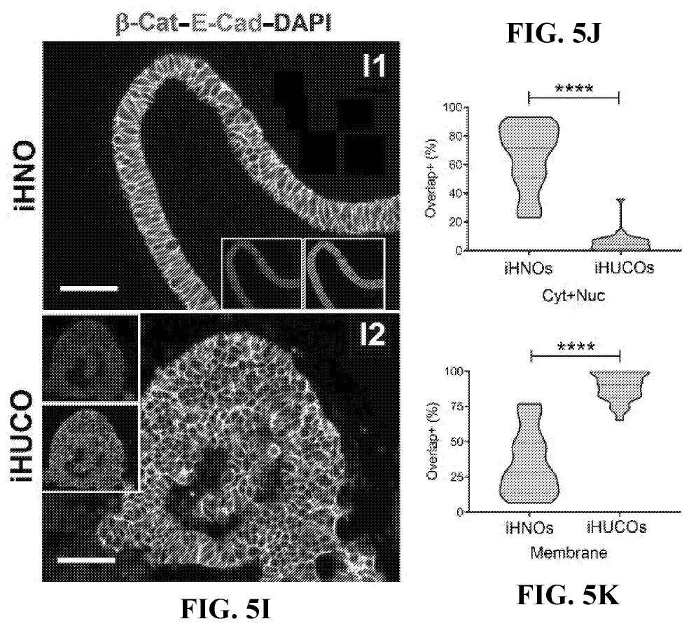

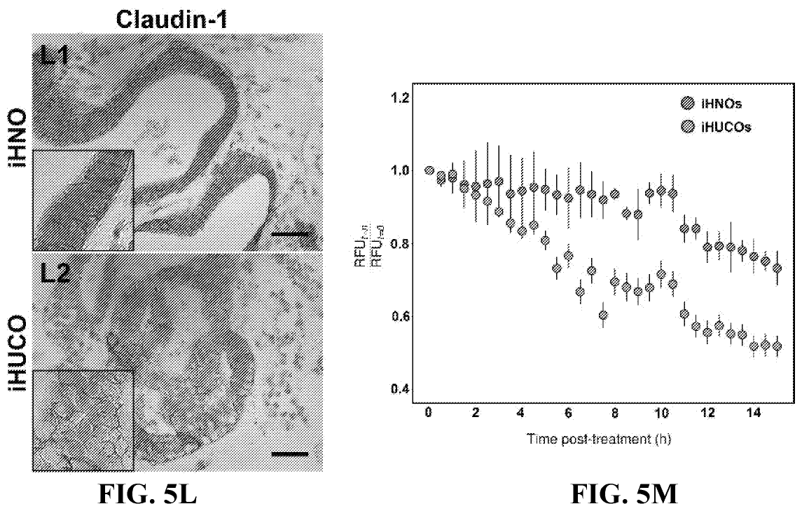

The unsupervised hierarchical clustering of all datasets (FIG. 4A) grouping organoids with parental fibroblasts led us to isolate the mesenchymal compartment of both iHNOs and iHUCOs (N=3 for each) for further analysis. Representative images of positive IF staining for Vimentin (a mesenchymal marker) in both non-IBD and UC mesenchyme along with the absence of the epithelial marker, CK19 is shown in FIG. 5A. Quantification of the percentage of cells expressing both proteins as well as α-SMA another marker for fibroblasts confirmed the mesenchymal identity of the isolates (FIG. 5B). Next, we used a cytokine array to compare the secretome of a subset of cytokine/chemokines in non-IBD and UC parental fibroblasts as well as isolated mesenchyme (FIG. 5C). The expression of GRO-α, GRO (α-β-c) and CXCL8 (IL-8) chemokines, which are all ligands of the CXCR1/2 receptor, was greater in both UC fibroblasts and UC mesenchyme than in non-IBD fibroblasts and mesenchyme (FIG. 5C).

The role of CXCL8, a multifunctional chemokine secreted by stromal cells in the inflammatory microenvironment, and its receptor CXCR1 have been extensively explored in tumorigenesis and progression of many types of cancer including colon cancer [44-47]. However, the role of CXCL8-induced signaling remains unclear in UC. The highlighted role of GPCR signaling in both epithelial and mesenchymal compartments of the iHUCOs (FIGS. 3E, 4C, and 4E) and the higher specificity of CXCR1 (binding only CXCL8 with high affinity) than CXCR2 (binding all ELRCXC chemokines with high affinity) [48] indicated the potential for interactions of CXCL8/CXCR1 as an inflammatory mediator and transducer of the G-protein—activating regulatory system [49].

Thus, we performed dual-immunofluorescent staining for the CXCL8 ligand and CXCR1 receptor in both iHNOs and iHUCOs (FIG. 5D-F). Quantification of the percentage of the cells expressing these proteins in epithelium and mesenchyme separately, confirmed that the expression of CXCL8 ligand and CXCR1 receptor were significantly greater in UC organoids (˜4-fold for each alone in parallel to co-localized CXCL8/CXCR1) than in non-IBD organoids (FIG. 5G, H).

One of the multiple downstream effects of the CXCL8/CXCR1 interaction is the regulation of RhoA as a CXCR1/CXCL8 signal transducer [50]. IHUCOs showed strong co-expression of CXCL8/CXCR1 (FIG. 5D-H) as well as the adherens junction complex signature via RhoA at the transcriptome and protein levels (FIGS. 2, 3E, and 4B2). Furthermore, individual IHC staining for E-cadherin and β-catenin (FIG. 2) revealed similar patterns of significant changes in the cellular localization of both proteins in UC and non-IBD organoids (FIG. 2A-J). Therefore, we studied the co-localization of E-cadherin and β-catenin by performing dual-immunofluorescent staining for proteins in iHNO and iHUCO organoids (FIG. 5I). Co-expression and tight association of both proteins as well as significant differences in their co-localization in UC and non-IBD organoids corresponded to that visualized in FIG. 2. Co-localization was mostly limited to the plasma membrane, highlighting a propensity towards adherens junctions rather than tight junctions in iHUCOs. In contrast, co-localization in the non-IBD condition extended to the cytoplasm and nucleus, indicating increased stability of protein expression in these subcellular locations. (FIGS. 5J, and K). Regarding the significant decrease of tight junction regulatory genes including OCLN and CLDN in the transcriptome of iHUCOs (but not iHNOs) vs. spheroids (FIG. 4D), we performed IHC for Claudin-1, a major constituent of the tight junction complexes responsible for the normal barrier function and prevention of the para-cellular small molecules diffusion in the epithelium [51]. The staining for both non-IBD and UC organoids showed a dramatic decrease of Claudin-1 expression in the epithelium of iHUCOs compared with iHNOs (FIG. 5L, S6G-Ctrl).

Moreover, to functionally confirm the adherens vs. tight junction signature in UC compared to non-IBD organoids, we used the recently described microinjection technique by Hill et al. [17] to measure and compare the epithelial barrier permeability for both UC and non-IBD organoids in real-time. Briefly, we microinjected organoids with fluorescently-labeled 4 kD dextran and imaged the organoids on an inverted microscope fitted with epifluorescent filters for a total of 15 hours. Real-time measurement of the barrier permeability showed significantly lower level of dye retention in the iHUCOs lumen (˜50% of real-time measurement timepoints) vs. iHNOs (FIG. 5M).

In sum, UC parental fibroblasts and iHUCOs-derived mesenchyme are similar as they both showed a dramatic increase in expression of CXCL8 and GRO chemokines. Both CXCL8 ligand and CXCR1 receptor were overexpressed in the epithelium and mesenchyme of UC vs. non-IBD organoids. We confirmed the co-expression and tight association between β-catenin and E-cadherin in organoids; in iHUCOs, both proteins co-localized predominantly in the plasma membrane whereas it extends to the cytoplasm and nucleus of iHNOs. Immunohistochemistry for the tight junction protein Claudin-1 along with our functional study of epithelial barrier permeability in organoids confirmed the compromise of tight junction in the epithelium of iHUCOs.

Repertaxin Attenuates the Progression of the Colitic Phenotype in iHUCOs In Vitro

The upregulation of the CXCL8 receptor pro-inflammatory interaction in iHUCOs (FIG. 5) led us to study the effect of repertaxin, a small molecule inhibitor of the CXCL8 receptor, on organoids development. In brief, we treated both UC and non-IBD organoids with repertaxin for 21 days, during their development from spheroids to organoids and compared their phenotypic characteristics to vehicle (Ctrl) organoids.

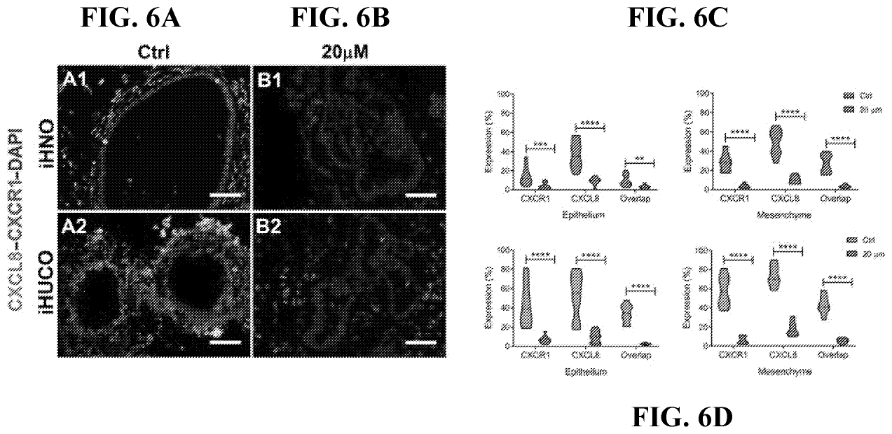

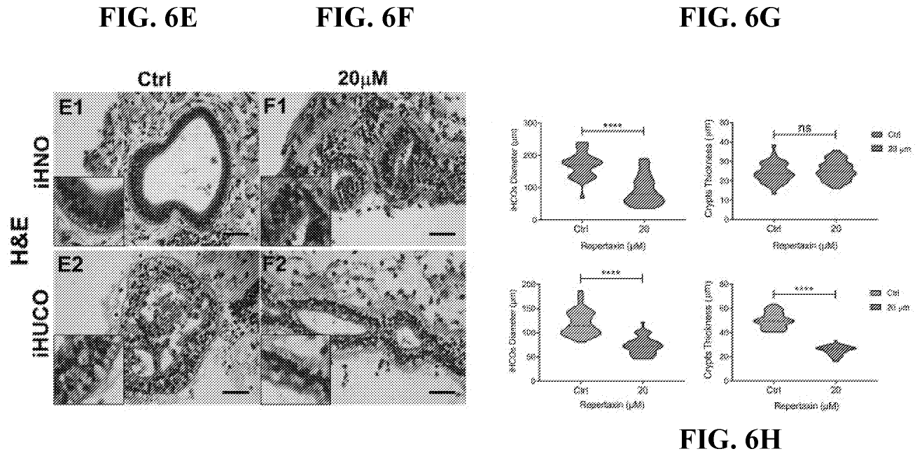

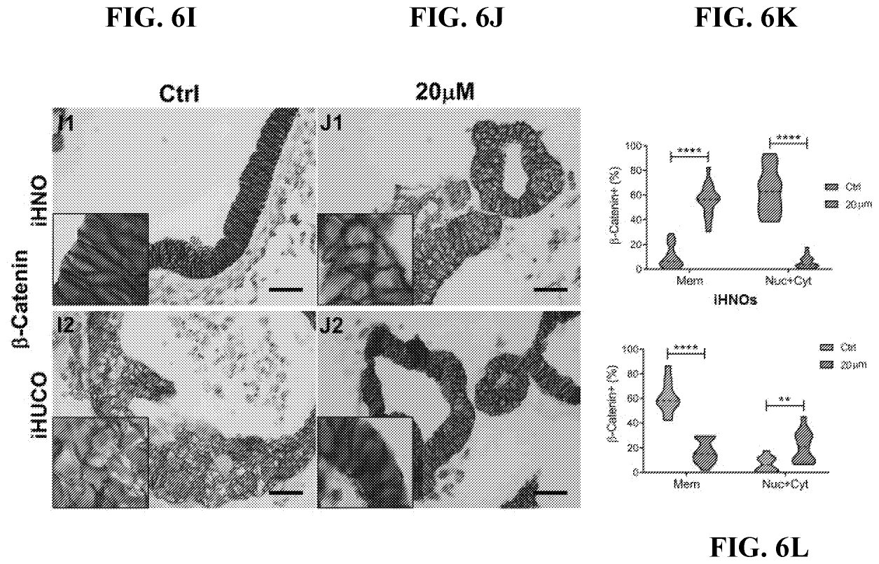

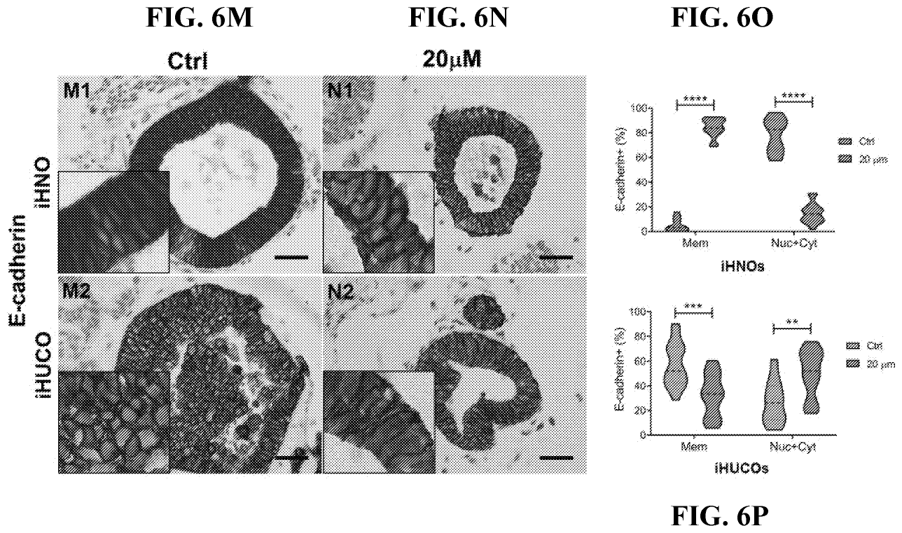

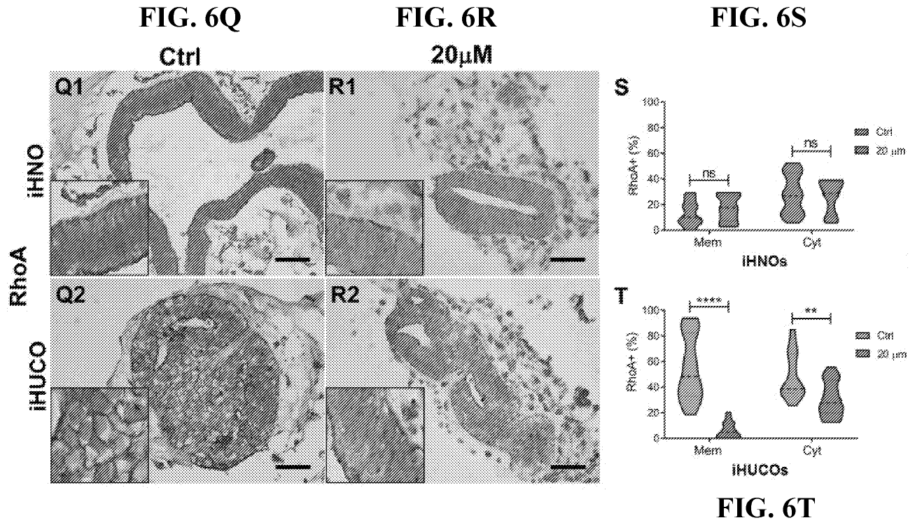

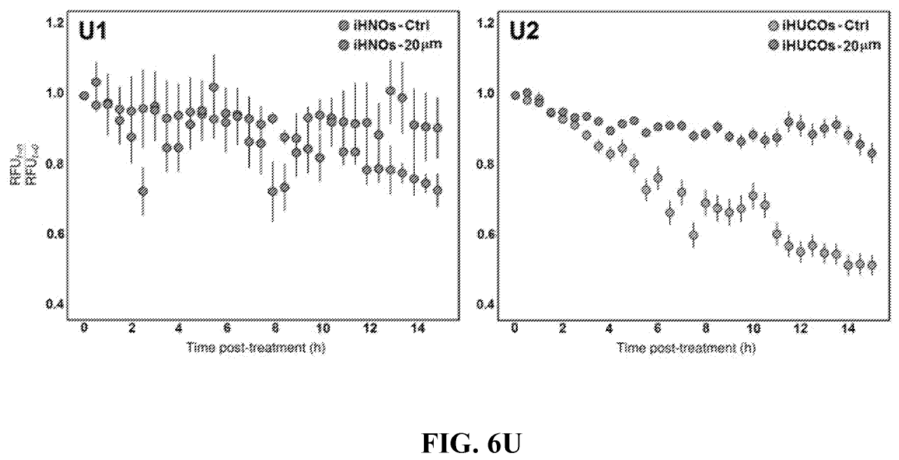

Expression of both CXCR1 and CXCL8 was significantly less in UC and non-IBD organoids with repertaxin than in the control organoids. (FIG. 6A, B). Quantification of this observation in iHUCOs confirmed an average of 7- and 9-fold lower expression of CXCR1 in the epithelium and mesenchyme, respectively. CXCL8 expression and the percentage of co-expression of CXCL8/CXCR1 were also significantly lower than control (FIG. 6C, D). Next, we examined the functional effect of repertaxin on the growth and morphology of non-IBD and UC organoids. In non-IBD, the treatment resulted in significantly smaller organoids but did not change their epithelial thickness (FIGS. 6E1, F1, and G). In contrast, repertaxin treatment resulted in significantly lower size and less thick epithelium in the iHUCOs (FIGS. 6E2, F2, and H). Consistent with this observation, IHC for the active proliferation marker, Ki67, confirmed significantly lower expression in the epithelium of repertaxin treated iHNOs, and even a greater effect on the aberrant proliferation of iHUCOs epithelium (Figure S6A-D).

IHC for β-catenin and E-cadherin proteins in the treated and control UC organoids showed that the cytoplasmic and nuclear expression of both β-catenin (FIGS. 6I2, J2, and L) and E-cadherin (FIGS. 6M2, N2, and P) were greater in repertaxin-treated UC organoids than in UC control organoids. On the other hand, the treatment of non-IBD organoids led to lower cytoplasmic and nuclear expression of β-catenin and E-cadherin than in the control, and significantly more cells with limited expression of β-catenin (FIGS. 6I1, J1, and K) and E-cadherin (FIGS. 6M1, N1, and O) on the plasma membrane. We also studied the effect of repertaxin on the expression pattern of RhoA. Although repertaxin did not significantly affect the expression of RhoA in iHNOs (FIGS. 6Q1, R1, and S), it caused less RhoA expression on the membrane (activated RhoA, by ˜5-fold) and cytoplasm of iHUCOs epithelium (FIG. 6Q2, R2, T).

Further, IHC analysis for the tight junction marker, Claudin-1, confirmed significantly more expression in iHUCOs after treatment with repertaxin (Figure S6E2, F2, and G). However, there was no significant changes in expression of Claudin-1 for treated and control iHNOs (Figure S6E1, F1, and G). To functionally test the effect of repertaxin treatment on the epithelial barrier permeability, we used the microinjection technique [17] to compare the rate of the dye release in treated and control organoids (FIG. 6U). Repertaxin did not have a significant effect on the epithelial permeability of non-IBD organoids (FIG. 6U1), but it significantly decreased the rate of permeability in the UC epithelial barrier (50% of real-time measurement timepoints) (FIG. 6U2).

We examined the inhibitory effect of repertaxin on the iHUCOs development by performing in situ hybridization for LGR5 and IHC for the p-YAP1 (Figure S6H). The expression patterns of LGR5 and p-YAP1 in treated iHUCOs more closely resembled the expression patterns in iHNOs (Figure S6H1, H2).

Therefore, the CXCR8 receptor inhibition by repertaxin significantly attenuated the progression of the colitic phenotype in iHUCOs in vitro. Repertaxin not only had a significant effect on the size and morphology of iHUCOs, but also modified the expression pattern of the proteins regulating the adherens junction complex, such that it was reversed to more closely resemble the iHNOs. We functionally validated these observations using the microinjection technique in real-time to show that while repertaxin treatment does not significantly affect the epithelial barrier permeability in non-IBD organoids, it sharply decreased the leakage in the UC epithelium.

Repertaxin Attenuates the Progression of the Colitic Phenotype in iHUCOs In Vivo

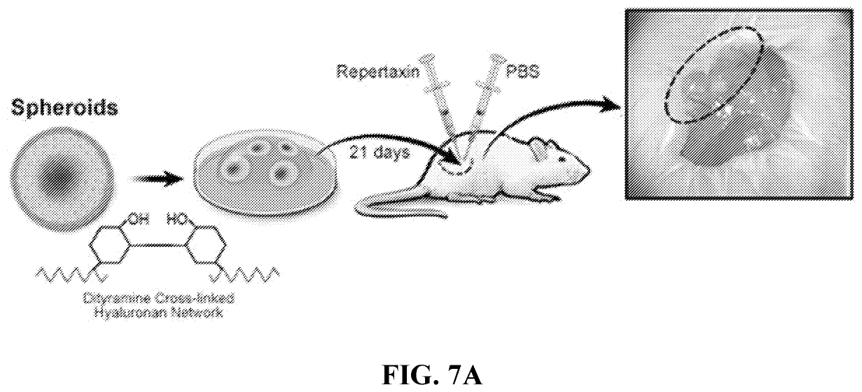

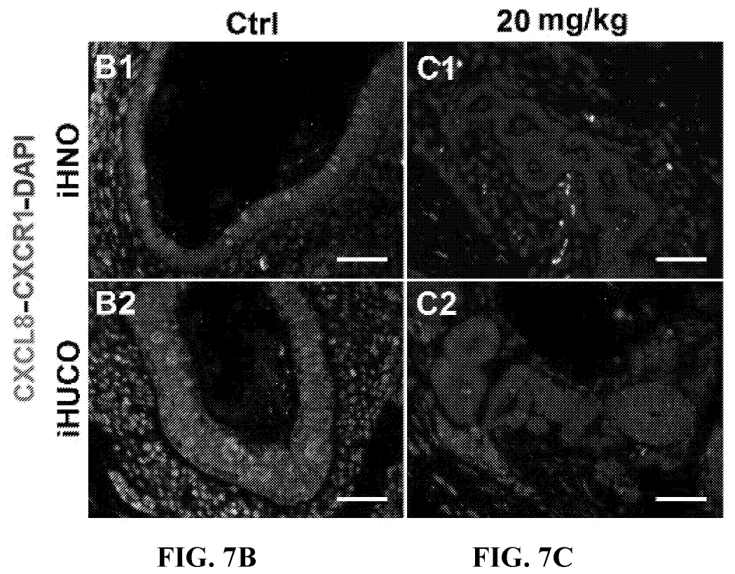

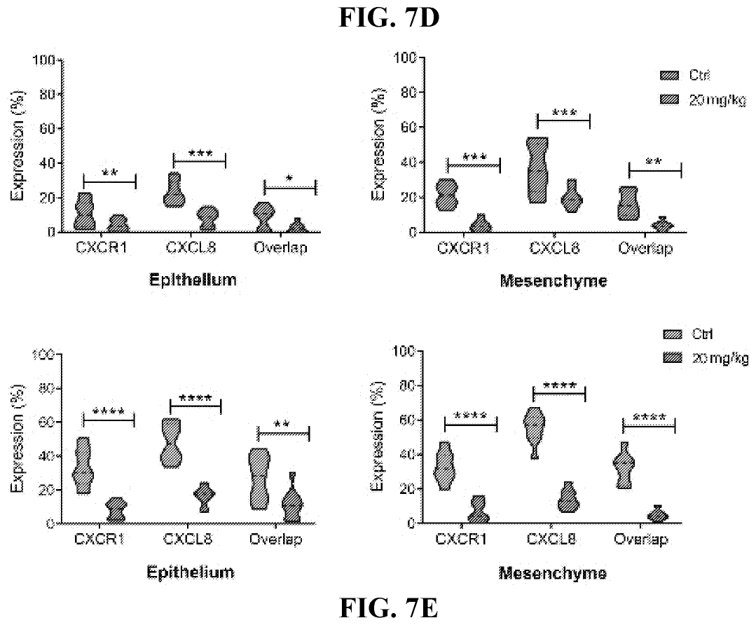

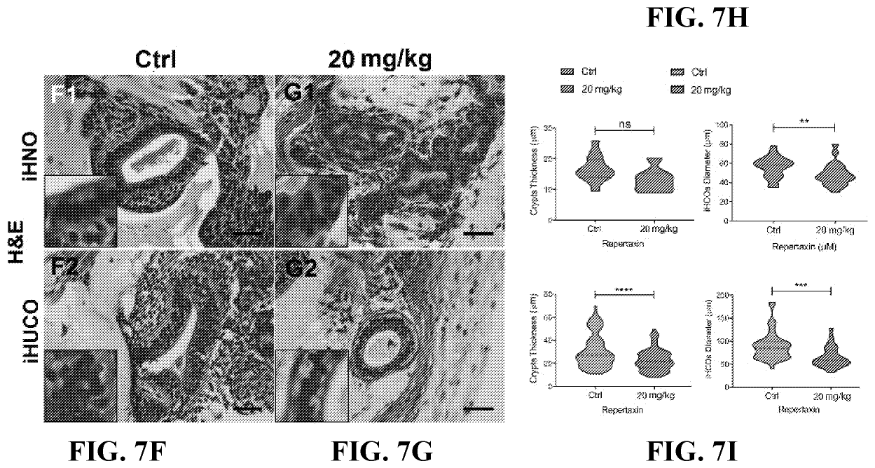

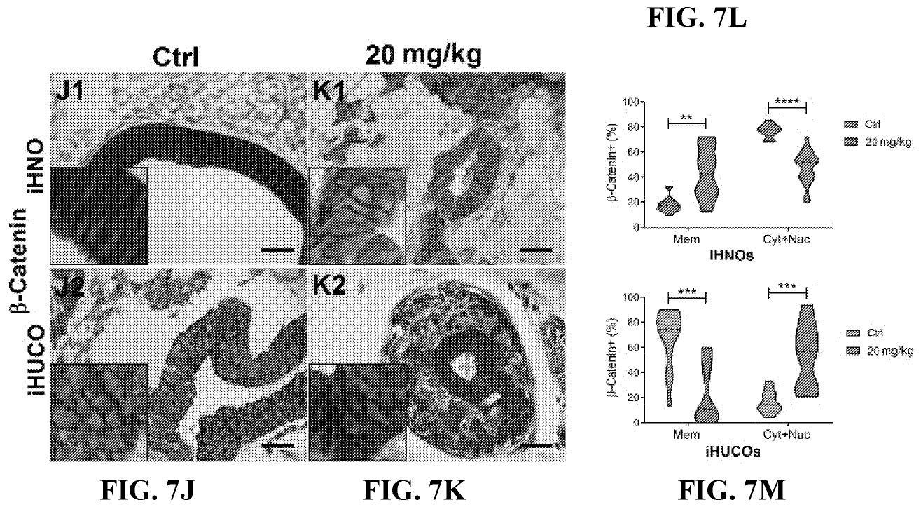

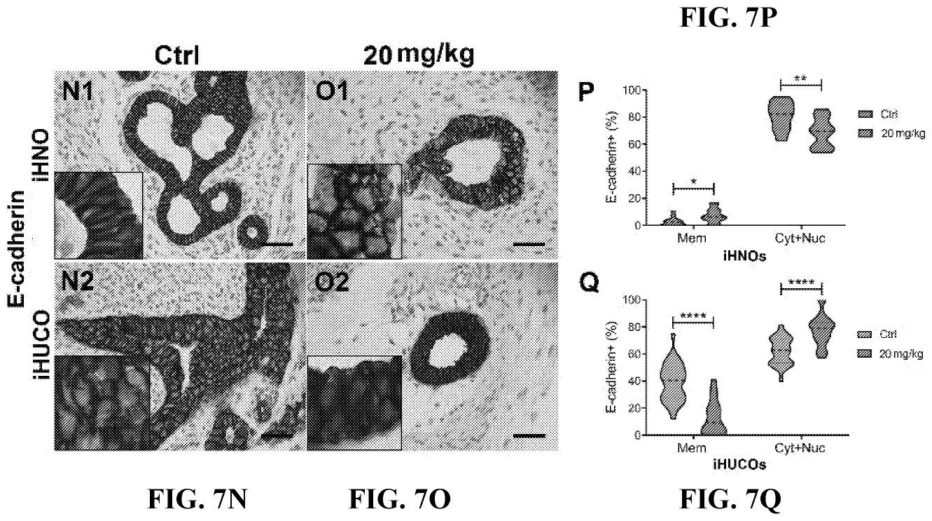

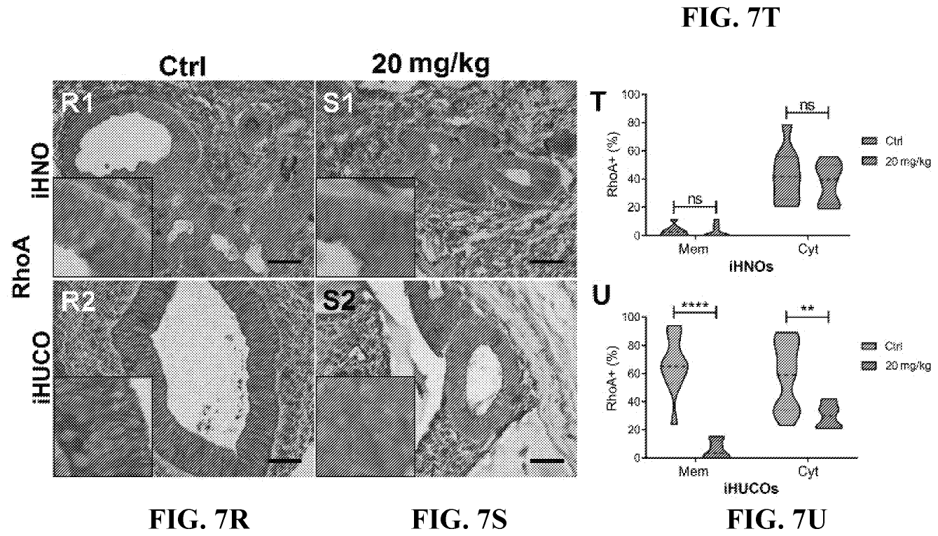

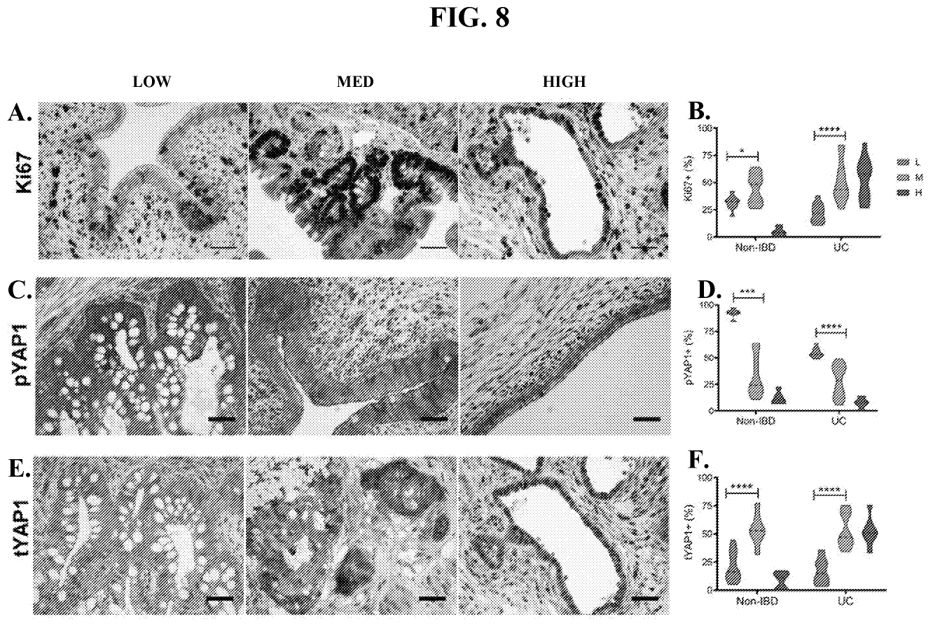

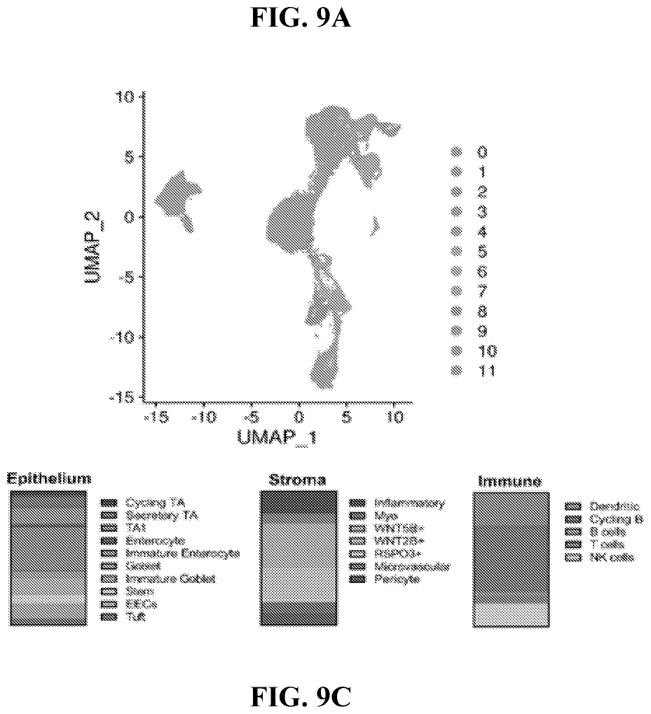

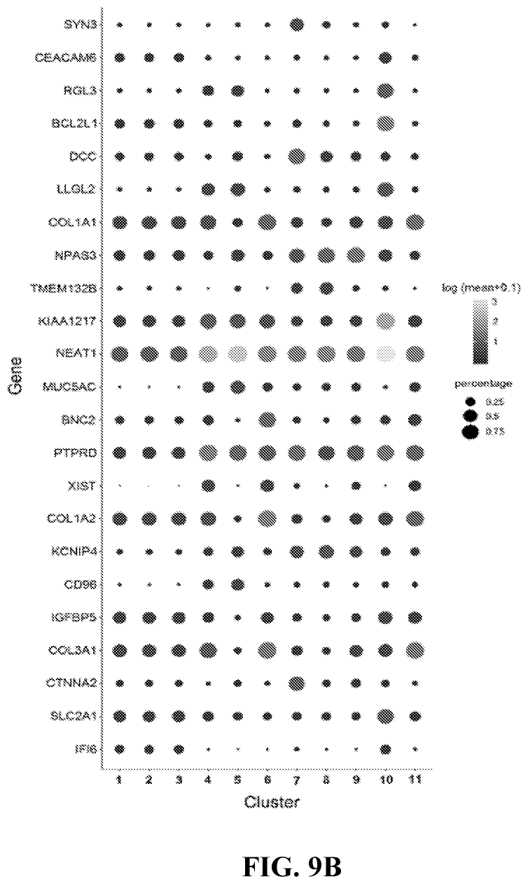

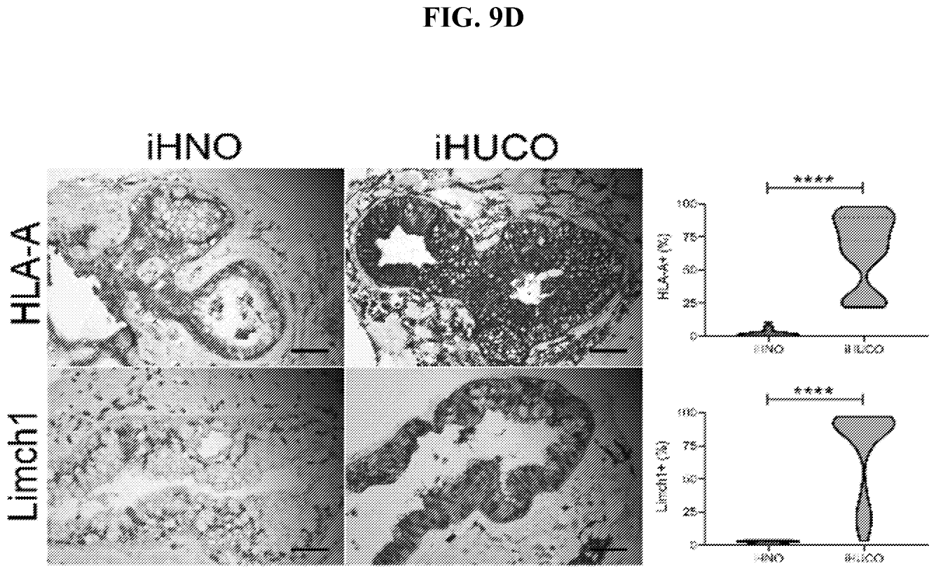

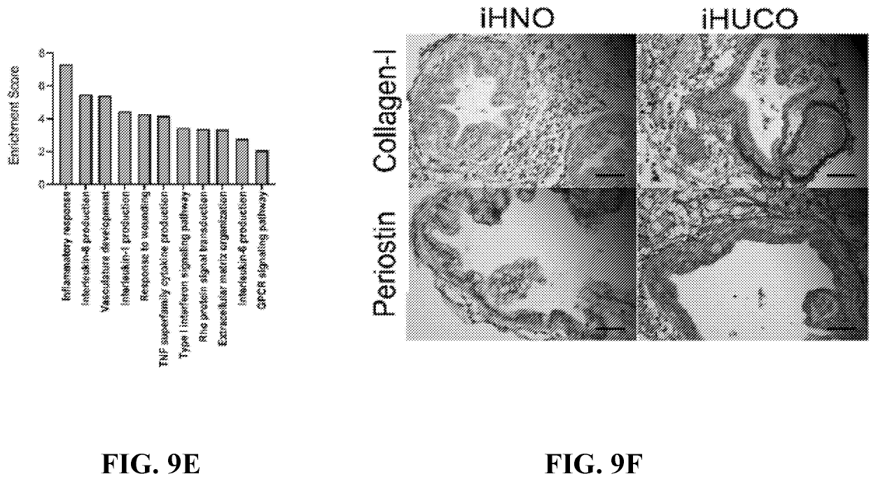

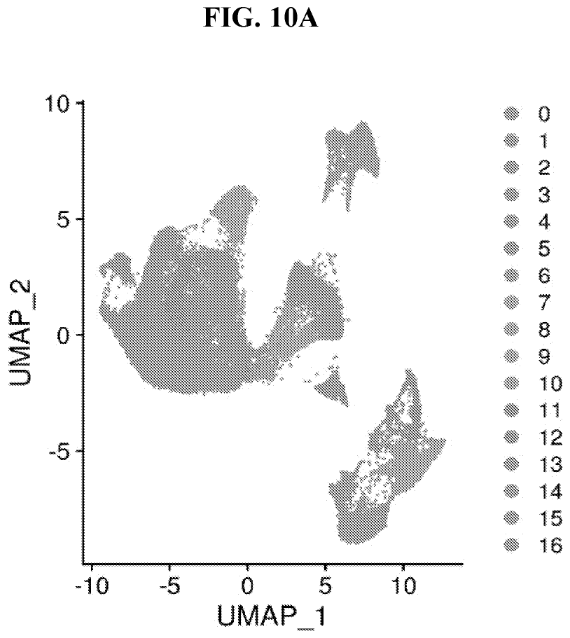

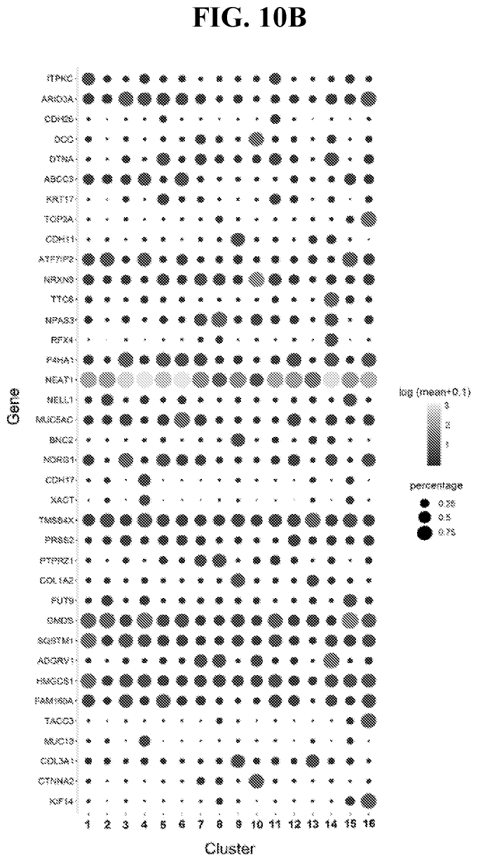

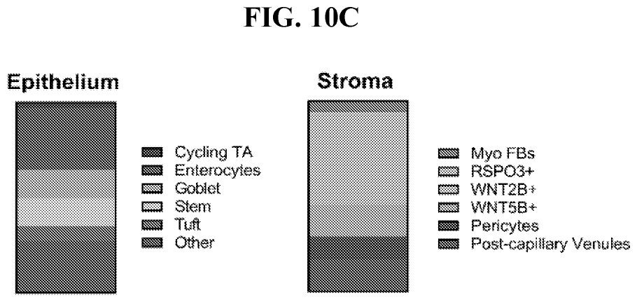

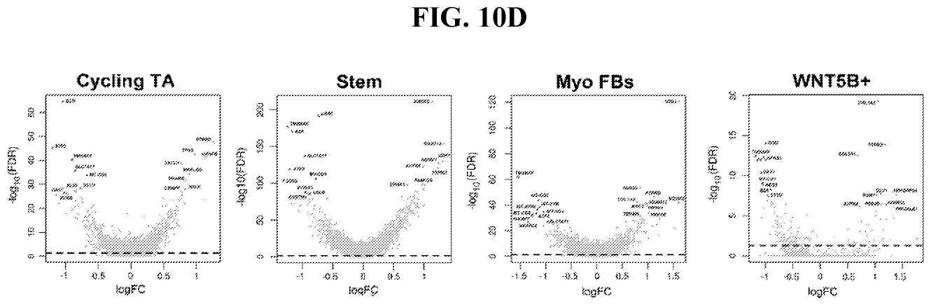

To test the significance of our repertaxin observations in vivo, we studied the effect of repertaxin on the developmental progression of spheroids to organoids, implanted subcutaneously in the dorsal flank of NSG mice (FIG. 7A, STAR Methods). In brief, we encapsulated the spheroids in TS-HA hydrogel beads and implanted the beads subcutaneously. Mice were then treated daily for 21 days with either 20 mg/kg repertaxin or PBS (control). The rate of growth was measured twice per week with calipers, and the volume calculated (STAR Methods). After 21 days, the overall calculated volume was significantly greater in the PBS than in the repertaxin-treated groups (Figure S6I); which was confirmed by H&E on the harvested beads after 21 days (FIG. 7F-I). In parallel to our in vitro observations, repertaxin treatment resulted in a significant less thick epithelium in the formed iHUCOs whereas it had no significant effect on iHNOs epithelial thickness (FIG. 7H, I). IHC for Ki67 confirmed that repertaxin treatment significantly reduced the proliferation rate in the epithelium of iHUCOs. However, it did not have a significant effect on the non-IBD epithelium (Figure S6J-M).

The harvested beads were also subjected to additional analyses. Consistent with our in vitro findings, CXCR1 and CXCL8 expression were less for both UC and non-IBD organoids treated with repertaxin (FIG. 7B-E). Also, the similar expression pattern as in vitro was present in the in vivo models for β-catenin and E-cadherin (FIG. 7J-Q). IHC analysis revealed that repertaxin treatment resulted in greater cytoplasmic and nuclear expression of both β-catenin (FIGS. 7J2-M) and E-cadherin (FIG. 7N2-Q) in iHUCOs than in the untreated control. In iHNOs, similar alternated patterns of expression as the in vitro study (higher rate of expression in membrane) were observed in both proteins (FIG. 7J1-L, 7N1-P). Also, consistent with our in vitro data, repertaxin treatment strongly decreased membranous and cytoplasmic expression of RhoA in iHUCOs whereas it did not have a significant effect on the RhoA expression in iHNOs (FIG. 7R-U). Similarly, repertaxin did not change the Claudin-1 expression in non-IBD organoids, but it significantly increased the expression of the protein in iHUCOs; indicating higher regulation of tight junctions in the UC epithelium (Figure S6N-Q)

These studies demonstrate that repertaxin treatment not only attenuated the colitic phenotype of iHUCOs in vitro but also had similar effects in vivo in term of morphology, size, and changes of the epithelial intercellular junction.

A substantial worldwide increase in the number of patients suffering from IBD has occurred; an 1.8 million (0.9%) US adults were estimated to have IBD in 1999 and that number rose to an estimated 3.1 million (1.3%) in 2015 [52-54]. Thus, an urgent need exists to advance current therapies with the ultimate goal of more effective treatment and preventive strategies. The complex nature of UC has made it challenging to develop a model to study colitis etiology. Moreover, despite the fact that the current therapeutic targets in IBD mainly focus on the suppression of immune responses [55], therapies often fail, thus highlighting the need to examine the role of both epithelial and mesenchymal compartments of the colon in disease development and progression.

In this report, we demonstrate the reprogramming of colonic fibroblasts isolated from UC patients can become iPSCs. We also show application of directed differentiation techniques to create an in vitro models of the UC colon (iHUCO) and non-IBD (iHNO). In contrast to the original report of the protocol for the development of small intestinal organoids (HIOs) [9], we demonstrate that the isolation of fibroblasts from UC and non-IBD colon was sufficient to retain the colonic identity in iHCOs. Notably, our model, includes both epithelial and mesenchymal compartments. It reflects the complexity and retains the colitic phenotype of the tissue of origin in vitro and in vivo in spite of reprogramming. Particularly, in the absence of additional environmental factors such as the microbiome, the intrinsic factors were sufficient to drive the UC.

We provided substantial evidence showing that iHUCO recapitulates primary tissue phenotypes at multiple levels including morphology, aberrant proliferation or differentiation, and absence of acidic mucus secretion as key features phenocopying the parental tissues. The presence of a leaky epithelial barrier, due to changes in the pattern of adherens and tight junctions at the epithelial intercellular junction in the iHUCOs demonstrates further recapitulation of the colitic signature. This simulation of phenotype may be a breakthrough in UC modeling, not only facilitating the exploration of strategies for personalized medicine but also investigating the mechanisms underlying the pathophysiology of human IBD and new therapeutic strategies in a less complex, more easily manipulated in vitro environment. In vivo, we verified the colon formation ability of our organoid models, making the models the prime candidate for use in colon regeneration (retaining the genetic background), and healing the damaged mucosa as a recent favorable approach in IBD treatment [56].

Although the autocrine and paracrine functions of CXCL8 chemokine and its receptor CXCR1 in the development of several types of cancer, including colorectal cancer, have been extensively studied [50, 57], the role of this inflammatory interaction in UC development and progression remains unclear. Using iHUCO, we provide the first evidence that shows overexpression of CXCL8/CXCR1 in UC disrupts canonical Wnt signaling regulation and results in a dysregulated adherens junction pattern in the iHUCO epithelial cells. Notably, CXCL8 lacks a murine orthologue, which highlights the gap in the murine-based models and the further functional importance of our model in identifying the role of CXCL8 receptor-mediated signaling in UC development and progression [58]. We also demonstrate the functionality of the models via responses to chemical perturbation by the CXCR8 receptor small molecule non-competitive inhibitor, repertaxin. Exposure of both in vitro and in vivo organoid cultures to repertaxin reduced the expression of CXCL8 ligand and CXCR1 receptor and attenuated several aspects of the colitic phenotype, including a disorganized epithelium, aberrant proliferation, and persistence of a leaky epithelial barrier, suggesting that the pro-inflammatory interaction of CXCR1-CXCL8 compromises the epithelial barrier, characteristic of colitis.

Our inducible organoid system provides a superior model to study the complexity of UC. It will permit the investigation of the developmental, pharmacologic, and genetic aspects of UC as well as epithelial-mesenchymal and intestinal microenvironmental interactions. Importantly, our protocols preserved the individual patient variations in disease. This preservation may originate from genetic predisposition and/or from epigenetic alterations in UC patients that are retained throughout iPSC reprogramming, providing a platform for future studies. Additionally, we may use the same approach to model other chronic inflammatory diseases such as Crohn's disease, the other main category of IBD, which has similar levels of complexity and challenges for modeling in vitro. Finally, we demonstrated overexpression of CXCL8 and its receptor in UC patient tissues, validating the significance of our functional studies. Thus, using repertaxin to block this interaction may be a promising therapeutic strategy to diminish the chronic inflammatory symptoms of ulcerative colitis.

### Example 2

### Exogenous Stiffness Results in Nuclear Translocation of Yap1 in an Induced Human Ulcerative Colitis-Derived Organoid Model

Colitis is a form of IBD characterized by chronic and relapsing episodes of bloody diarrhea. Repeated colitic attacks results in fibrosis and strictures. Over time, colitic epithelia is at increased risk for dysplasia and cancer. No previous 3D in vitro models of human colitis include both the epithelia and the mesenchyme.

### Methods

Yamanaka factors were used to reprogram NL and UC isolated fibroblasts into induced pluripotent stem cells (iPSCs) followed by directed differentiation to the colonic organoids. To mimic the intraabdominal microenvironment with correlating levels of exogenous stiffness, the resulting NL and UC organoids were encapsulated into TS-HA hydrogel beads with low (<2 kPa), medium (4-6 kPa), and high (>8 kPa) moduli, and then transplanted into the omentum of NOD-SCID IL2^{γ} receptor null mice. At harvest, immunohistochemistry compared proliferation (Ki67), Nuclear total Yap1 (tYAP1) and cytoplasmic phosphorylated-Yap1 (pYAP1, Serine127) stained cells were enumerated.

### Results

Induced human non-IBD (iHN) and UC (iHUC) organoids encapsulated in TS-HA hydrogel beads transplanted in the omentum, phenocopied the primary tissues regarding morphology, proliferation, and hindgut markers. Notably, with increased intraabdominal mechanical stiffness, only the UC derived iHIOs were able to proliferate and form the cystic organoids (FIG. 8). In parallel, increasing levels of nuclear total Yap1 were present with increasing stiffness in the UC-derived organoids (p<0.0001). However, by increasing the moduli pYAP1 was decreased in iHUCOs.

### CONCLUSION

The induced human non-IBD (iHN) and UC (iHUC) organoids phenocopy their tissues of origin and are responsive to both local microenvironmental cues as well as to intraabdominal cues. As such, these models can serve as avatars for precision medicine.

### REFERENCES

1. Calixto, et al., Arq Gastroenterol, 2018. 55(3): p. 202-207.
2. Al-Ghadban, et al., Sci Rep, 2016. 6: p. 29783.
3. Kaiko, G. E., et al., PAI-1 augments mucosal damage in colitis. Sci Transl Med, 2019. 11(482).
4. MacManus, C. F., et al., Interleukin-8 signaling promotes translational regulation of cyclin D in androgen-independent prostate cancer cells. Mol Cancer Res, 2007. 5(7): p. 737-48.
5. Luppi, F., et al., Interleukin-8 stimulates cell proliferation in non-small cell lung cancer through epidermal growth factor receptor transactivation. Lung Cancer, 2007. 56(1): p. 25-33.
6. Kamohara, H., et al., Induction of interleukin-8 (CXCL-8) by tumor necrosis factor-alpha and leukemia inhibitory factor in pancreatic carcinoma cells: Impact of CXCL-8 as an autocrine growth factor. Int J Oncol, 2007. 31(3): p. 627-32.
7. Yasumoto, K., et al., Tumor necrosis factor alpha and interferon gamma synergistically induce interleukin 8 production in a human gastric cancer cell line through acting concurrently on AP-1 and NF-kB-like binding sites of the interleukin 8 gene. J Biol Chem, 1992. 267(31): p. 22506-11.
8. Schraufstatter, I. U., J. Chung, and M. Burger, IL-8 activates endothelial cell CXCR1 and CXCR2 through Rho and Rac signaling pathways. Am J Physiol Lung Cell Mol Physiol, 2001. 280(6): p. L1094-103.
9. Moore, K. A. and I. R. Lemischka, Stem cells and their niches. Science, 2006. 311(5769): p. 1880-5.
10. Clevers, H., Wnt/beta-catenin signaling in development and disease. Cell, 2006. 127(3): p. 469-80.
11. Logan, C. Y. and R. Nusse, The Wnt signaling pathway in development and disease. Annu Rev Cell Dev Biol, 2004. 20: p. 781-810.
12. Gong, Y., C. Mo, and S. E. Fraser, Planar cell polarity signalling controls cell division orientation during zebrafish gastrulation. Nature, 2004. 430(7000): p. 689-93.
13. Habas, R., I. B. Dawid, and X. He, Coactivation of Rac and Rho by Wnt/Frizzled signaling is required for vertebrate gastrulation. Genes Dev, 2003. 17(2): p. 295-309.
14. Sato, T., et al., Long-term expansion of epithelial organoids from human colon, adenoma, adenocarcinoma, and Barrett's epithelium. Gastroenterology, 2011. 141(5): p. 1762-72.
15. VanDussen, K. L., et al., Development of an enhanced human gastrointestinal epithelial culture system to facilitate patient-based assays. Gut, 2015. 64(6): p. 911-20.
16. Goyal, N., et al., Animal models of inflammatory bowel disease: a review. Inflammopharmacology, 2014. 22(4): p. 219-33.
17. Takahashi, K. and S. Yamanaka, Induction of pluripotent stem cells from mouse embryonic and adult fibroblast cultures by defined factors. Cell, 2006. 126(4): p. 663-76.
18. McCracken, K. W., et al., Generating human intestinal tissue from pluripotent stem cells in vitro. Nat Protoc, 2011. 6(12): p. 1920-8.
19. Carpentino, J. E., et al., Aldehyde dehydrogenase-expressing colon stem cells contribute to tumorigenesis in the transition from colitis to cancer. Cancer Res, 2009. 69(20): p. 8208-15.
20. DeRoche, T. C., S. Y. Xiao, and X. Liu, Histological evaluation in ulcerative colitis. Gastroenterol Rep (Oxf), 2014. 2(3): p. 178-92.
21. Gibson, P., et al., Colonic epithelium is diffusely abnormal in ulcerative colitis and colorectal cancer. Gut, 1995. 36(6): p. 857-63.
22. Pelaseyed, T., et al., The mucus and mucins of the goblet cells and enterocytes provide the first defense line of the gastrointestinal tract and interact with the immune system. Immunol Rev, 2014. 260(1): p. 8-20.
23. Dorofeyev, A. E., et al., Mucosal barrier in ulcerative colitis and Crohn's disease.

Gastroenterol Res Pract, 2013. 2013: p. 431231.
24. Wells, J. M., Developmental biology. Regional identity of gut stem cells—one gene to rule them all. Nat Rev Gastroenterol Hepatol, 2015. 12(3): p. 125-6.
25. Suh, E. and P. G. Traber, An intestine-specific homeobox gene regulates proliferation and differentiation. Mol Cell Biol, 1996. 16(2): p. 619-25.
26. Coskun, M., et al., TNF-alpha-induced down-regulation of CDX2 suppresses MEP1A expression in colitis. Biochim Biophys Acta, 2012. 1822(6): p. 843-51.
27. Coskun, M., The role of CDX2 in inflammatory bowel disease. Dan Med J, 2014. 61(3): p. B4820.
28. Munera, J. O., et al., Differentiation of Human Pluripotent Stem Cells into Colonic Organoids via Transient Activation of BMP Signaling. Cell Stem Cell, 2017. 21(1): p. 51-64. e6.
29. Finkbeiner, S. R., et al., Transcriptome-wide analysis reveals hallmarks of human intestine development and maturation in vitro and in vivo. Stem cell reports, 2015. 4(6): p. 1140-1155.
30. Citi, S., Intestinal barriers protect against disease. Science, 2018. 359(6380): p. 1097-1098.
31. Odenwald, M. A. and J. R. Turner, The intestinal epithelial barrier: a therapeutic target? Nat Rev Gastroenterol Hepatol, 2017. 14(1): p. 9-21.
32. Clayburgh, D. R., L. Shen, and J. R. Turner, A porous defense: the leaky epithelial barrier in intestinal disease. Lab Invest, 2004. 84(3): p. 282-91.
33. Luissint, A. C., C. A. Parkos, and A. Nusrat, Inflammation and the Intestinal Barrier: Leukocyte-Epithelial Cell Interactions, Cell Junction Remodeling, and Mucosal Repair. Gastroenterology, 2016. 151(4): p. 616-32.
34. Valenta, T., G. Hausmann, and K. Basler, The many faces and functions of beta-catenin. Embo j, 2012. 31(12): p. 2714-36.
35. Harris, T. J. and U. Tepass, Adherens junctions: from molecules to morphogenesis. Nat Rev Mol Cell Biol, 2010. 11(7): p. 502-14.
36. Cizelsky, W., et al., The Wnt/JNK signaling target gene alcam is required for embryonic kidney development. Development, 2014. 141(10): p. 2064-74.
37. Zhao, B., et al., Inactivation of YAP oncoprotein by the Hippo pathway is involved in cell contact inhibition and tissue growth control. Genes Dev, 2007. 21(21): p. 2747-61.
38. De, A., Wnt/Ca2+ signaling pathway: a brief overview. Acta Biochim Biophys Sin (Shanghai), 2011. 43(10): p. 745-56.
39. Finkbeiner, S. R., et al., Generation of tissue-engineered small intestine using embryonic stem cell-derived human intestinal organoids. Biol Open, 2015. 4(11): p. 1462-72.
40. Shannon, P., et al., Cytoscape: a software environment for integrated models of biomolecular interaction networks. Genome Res, 2003. 13(11): p. 2498-504.
41. Supek, F., et al., REVIGO summarizes and visualizes long lists of gene ontology terms.

PLoS One, 2011. 6(7): p. e21800.
42. Rajagopalan, L. and K. Rajarathnam, Ligand selectivity and affinity of chemokine receptor CXCR1. Role of N-terminal domain. J Biol Chem, 2004. 279(29): p. 30000-8.
43. Ha, H., B. Debnath, and N. Neamati, Role of the CXCL8-CXCR1/2 Axis in Cancer and Inflammatory Diseases. Theranostics, 2017. 7(6): p. 1543-1588.
44. Liu, Q., et al., The CXCL8-CXCR1/2 pathways in cancer. Cytokine Growth Factor Rev, 2016. 31: p. 61-71.
45. Buckley, A. and J. R. Turner, Cell Biology of Tight Junction Barrier Regulation and Mucosal Disease. Cold Spring Harb Perspect Biol, 2018. 10(1).
46. Hill, D. R., et al., Real-time Measurement of Epithelial Barrier Permeability in Human

Intestinal Organoids. J Vis Exp, 2017(130).
47. Gasparetto, M. and G. Guariso, Highlights in IBD Epidemiology and Its Natural History in the Paediatric Age. Gastroenterol Res Pract, 2013. 2013: p. 829040.
48. Ghione, S., et al., Dramatic Increase in Incidence of Ulcerative Colitis and Crohn's Disease (1988-2011): A Population-Based Study of French Adolescents. Am J Gastroenterol, 2018. 113(2): p. 265-272.
49. Dahlhamer, J. M., et al., Prevalence of Inflammatory Bowel Disease Among Adults Aged >/=18 Years—United States, 2015. MMWR Morb Mortal Wkly Rep, 2016. 65(42): p. 1166-1169.
50. Perse, M. and A. Cerar, Dextran sodium sulphate colitis mouse model: traps and tricks. J Biomed Biotechnol, 2012. 2012: p. 718617.
51. Waugh, D. J. and C. Wilson, The interleukin-8 pathway in cancer. Clin Cancer Res, 2008. 14(21): p. 6735-41.
52. Becker, M. D., et al., Reduced leukocyte migration, but normal rolling and arrest, in interleukin-8 receptor homologue knockout mice. Invest Ophthalmol Vis Sci, 2000. 41(7): p. 1812-7.
53. Nakamura, T. and T. Sato, Advancing Intestinal Organoid Technology Toward Regenerative Medicine. Cell Mol Gastroenterol Hepatol, 2018. 5(1): p. 51-60.
54. Yui, S., et al., YAP/TAZ-Dependent Reprogramming of Colonic Epithelium Links ECM Remodeling to Tissue Regeneration. Cell Stem Cell, 2018. 22(1): p. 35-49.e7.
55. Spence J R, Mayhew C N, Rankin S A, Kuhar M F, Vallance J E, Tolle K, Hoskins E E, Kalinichenko V V, Wells S I, Zorn A M, Shroyer N F, Wells J M. Directed differentiation of human pluripotent stem cells into intestinal tissue in vitroNature. 2011 Feb. 3; 470(7332):105-9.
56. Liu, Q., et al., The CXCL8-CXCR1/2 pathways in cancer. Cytokine Growth Factor Rev, 2016. 31: p. 61-71.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of generating colitic induced human colonic organoids (iHCOS) in comprising:
a) contacting a population of colonic fibroblasts from a human subject with inflammatory bowel disease (IBD) with: i) one or more expression vectors encoding IPSC reprogramming factors, or ii) RNAs encoding said IPSC reprogramming factors; to generate induced pluripotent stem cells (IPSCs),
b) contacting said IPSCs with a transforming growth factor beta pathway agonist to generate definitive endoderm;
c) contacting said definitive endoderm with a WNT signaling pathway agonist, a WNT/FGF signaling pathway agonist, a FGF signaling pathway agonist, or a combination thereof, thereby generating colitic induced human spheroids; and
d) culturing said spheroids in culture media with at least one of the following: Respondin1, Noggin, EGF, retinoic acid, and a BMP inhibitor, thereby generating colitic induced human colitic organoids (iHCOS).

2. The method of claim 1, wherein said IBD is ulcerative colitis.

3. The method of claim 1, wherein said IBD is Crohn's disease.

4. The method of claim 1, wherein said transforming growth factor beta pathway agonist comprises Activin A.

5. The method of claim 1, wherein said FGF signaling pathway agonist is FGF4.

6. The method of claim 1, wherein said WNT pathway agonist is WNT3a.

7. A composition comprising: a colitic induced human colitic organoid (iHCO), wherein said iHCO comprises an epithelial compartment and mesenchymal compartment, and provides at least one feature of IBD patient tissue.

8. The composition of claim 8, wherein said at least one feature comprises a leaky epithelial barrier.

9. The composition of claim 8, wherein said at least one feature is selected from the group consisting of: disorganization of said epithelium compartment, elevated expression of CXCL8, and elevated expression of CXCR1.

10. The composition of claim 7, wherein said composition further comprises growth media, a hydrogel, and/or one or more candidate IBD treating compounds.

11. The composition of claim 7, wherein said composition is located in vitro.

12. The composition of claim 7, wherein said IBD tissue comprises ulcerative colitis tissue.

13. The composition of claim 7, wherein said IBD tissue comprises Crohn's disease tissue.

14. A composition comprising: a colitic induced human spheroid.

15. The composition of claim 14, wherein said composition further comprises growth media, a hydrogel, and/or one or more candidate IBD treating compounds.

16. A kit or system comprising:
a) colitic induced human colitic organoid (iHCO) and/or a colitic induced human spheroid; and
b) a candidate IBD treating compound.

17. A method of screening candidate IBD treating compounds in vitro comprising:
a) contacting a colitic induced human colitic organoid (iHCO) with a candidate IBD treating compound, wherein said iHCO comprises an epithelial compartment and mesenchymal compartment, and provides at least one feature of IBD patient tissue; and
b) determining if said contacting causes said at least one feature of IBD patient tissue to be more like non-IBD tissue.

18. The method of claim 17, wherein said iHCO is derived from a colonic fibroblast from a human subject with IBD.

19. The method of claim 18, wherein said contacting is found to cause said at least one feature of IBD patient tissue to be more like non-IBD tissue, and wherein the method further comprises treating said subject with said candidate IBD treating compound.

20. The method of claim 17, wherein said IBD patient tissue comprises Ulcerative Colitis patient tissue.

21. The method of claim 17, wherein said IBD patient tissue comprises Crohn's disease patient tissue.

22. A method of screening candidate IBD treating compounds in vivo comprising:
a) implanting a composition into a test animal, wherein said composition comprises: a colitic induced human colitic organoid (iHCO) and/or a colitic induced human spheroid (iHS); and
b) administering a candidate IBD treatment compound to said test animal.

23. The method of claim 22, further comprising: c) examining said iHCO and/or iHS for changes.

24. The method of claim 22, wherein said composition comprises a hydrogel surrounding said iHCO and/or iHS.
